(19) United States
(12) Patent Application Publication
(10) Pub. No.: US 2025/0179540 A1
(43) Pub. Date: Jun. 5, 2025

(54) IMPROVED CYSTEINE-PRODUCING STRAINS

(71) Applicant: WACKER CHEMIE AG, Munich (DE)

(72) Inventors: Rupert PFALLER, Munich (DE); Andrea HEDLER, Munich (DE)

(73) Assignee: WACKER CHEMIE AG, Munich (DE)

(21) Appl. No.: 18/841,441

(22) PCT Filed: Mar. 1, 2022
(86) PCT No.: PCT/EP2022/055177

§ 371 Date: Aug. 26, 2024

(51) Int. Cl. C12P 13/12; C12N 15/70; C12P 17/14

(52) U.S. Cl. CPC C12P 13/12; C12N 15/70; C12P 17/14; C12N 2800/101

(57) ABSTRACT

A microorganism strain has a deregulated cysteine biosynthesis pathway that is thereby suitable for fermentative production of at least one substance selected from L-cysteine, L-cystine and thiazolidine. The relative expression of the crp gene is reduced in relation to the expression of the crp gene having a wild-type promoter sequence as a result of mutation of the crp promoter sequence.

The invention relates to a microorganism strain comprising a deregulated cysteine biosynthesis pathway that is thereby suitable for fermentative production of at least one substance selected from L-cysteine, L-cystine and thiazolidine, characterized in that the relative expression of the crp gene is reduced in relation to the expression of the crp gene having a wild-type promoter sequence as a result of mutation of the crp promoter sequence. A particular advantage is that said microorganism strain forms an increased amount of a substance selected from L-cysteine, L-cystine and thiazolidine compared to the corresponding microorganism strain with expression of the crp gene having a wild-type promoter. The invention therefore also provides a process for producing at least one compound selected from L-cysteine and its derivatives L-cystine and thiazolidine using said microorganism strain.

Cysteine, abbreviated Cys or C, is an α-amino acid having the side chain —CH₂—SH. Since the naturally occurring enantiomeric form is L-cysteine and since only this is a proteinogenic amino acid, it is in the context of the present invention L-cysteine that is meant when the term cysteine is used without a descriptor. Oxidation of the sulfhydryl groups can result in two cysteine residues together forming a disulfide bond, with consequent formation of cystine, to which the same statement applies, i.e., in the absence of a descriptor it is the L-enantiomer (or L-cystine, or (R,R)-3,3′-dithiobis(2-aminopropionic acid)) that is meant in the present invention. L-cysteine is a semi-essential amino acid for humans, since it can be formed from the amino acid methionine. Thiazolidine refers to the compound 2-methyl-2,4-thiazolidinedicarboxylic acid, an adduct of cysteine and pyruvate (EP 0 885 962 B1).

In all organisms, cysteine occupies a key position in sulfur metabolism and is used in the synthesis of proteins, glutathione, biotin, lipoic acid, thiamine, taurine, methionine and other sulfur-containing metabolites. Moreover, L-cysteine serves as a precursor for the biosynthesis of coenzyme A.

The biosynthesis of cysteine has been studied in detail in bacteria, especially in enterobacteria. A summary of cysteine biosynthesis can be found in Wada and Takagi, Appl. Microbiol. Biotechnol. (2006) 73:48-54.

The amino acid L-cysteine is of economic importance. It is used for example as a food additive (particularly in the baking industry), as a raw material in cosmetics, and as a starting material for the production of active pharmaceutical ingredients (particularly N-acetylcysteine and S-carboxymethylcysteine).

Besides classic preparation of cysteine by means of extraction from keratin-containing material such as hair, bristles, horns, hoofs and feathers, or by means of biotransformation through enzymatic conversion of precursors, there is also a process for fermentative production of cysteine. The prior art with regard to the fermentative preparation of cysteine using microorganisms is disclosed, for example, in EP 0 858 510 B1, EP 0 885 962 B1, EP 1 382 684 B1, EP 1 220 940 B2, EP 1 769 080 B1, EP 2 138 585 B1 and WO 2021/259491. The bacterial host organisms that are used include strains of the genus Corynebacterium and members of the Enterobacteriaceae family, such as Escherichia coli or Pantoea ananatis.

Although wild-type host organisms with no further modification contain a cysteine biosynthesis pathway (see, for example, the KEGG pathway database: “Cysteine and methionine metabolism”), said pathway is regulated such that the amount of cysteine produced is only an amount necessary for cell growth. As described, for example, in a review article by Wada and Takagi 2006 (see above), cysteine biosynthesis is regulated in WT strains by so-called feedback inhibition of key enzymes. For instance, L-serine inhibits the SerA enzyme 3-phosphoglycerate dehydrogenase and L-cysteine inhibits the CysE enzyme serine O-acetyltransferase. SerA and CysE are both enzymes of the cysteine biosynthesis pathway and their feedback inhibition by L-serine and L-cysteine, respectively, prevents more cysteine from being formed than is required by the cell. Such wild-type strains do not produce any detectable cysteine, as disclosed, for example, for the strain E. coli K12 W3110 in Table 2 of the present invention, and are therefore unsuitable for production of cysteine despite the presence of a cysteine biosynthesis pathway. A wild-type microorganism strain becomes suitable for cysteine production by deregulation of the cysteine biosynthesis pathway. To produce microorganism strains which have deregulated cysteine biosynthesis and which are distinguished by improved cysteine production, various methods are available. Besides the classic approach of using mutation and selection to arrive at improved cysteine producers, specific genetic modifications have also been made to the strains in order to achieve effective overproduction of cysteine.

For instance, the introduction of a cysE allele which encodes a serine O-acetyltransferase having reduced feedback inhibition by cysteine led to an increase in cysteine production (EP 0 858 510 B1; Nakamori et al., Appl. Env. Microbiol. (1998) 64:1607-1611). A feedback-resistant CysE enzyme largely decouples the formation of O-acetyl-L-serine, the direct precursor of cysteine, from the cysteine level in the cell.

O-Acetyl-L-serine is formed from L-serine and acetyl-CoA. Therefore, it is of great importance to provide L-serine in sufficient quantity for cysteine production. This can be achieved by introducing a serA allele encoding a 3-phosphoglycerate dehydrogenase having reduced feedback inhibition by L-serine. As a result, the formation of 3-phosphohydroxypyruvate, a biosynthetic precursor of L-serine, is largely decoupled from the L-serine level in the cell. Examples of such SerA enzymes are described in EP 0 620 853 B1 and EP 1 496 111 B1. Alternatively, Bell et al., Eur. J. Biochem. (2002) 269:4176-4184 disclose modifications to the serA gene to deregulate enzyme activity.

Increasing the transport of cysteine out of the cell is another way to increase the product yield in the medium. This can be achieved by overexpressing so-called efflux genes. Said genes encode membrane-bound proteins which mediate the export of cysteine out of the cell. Various efflux genes for cysteine export have been described (EP 0 885 962 B1, EP 1 382 684 B1). The export of cysteine out of the cell into the fermentation medium has several advantages:
1) L-cysteine is continuously withdrawn from the intracellular reaction equilibrium, with the result that the level of this amino acid in the cell is kept low and the feedback inhibition of sensitive enzymes by L-cysteine thus ceases:
   (1) L-cysteine (intracellular)<->L-cysteine (medium)
2) The L-cysteine secreted into the medium is oxidized to form the disulfide L-cystine in the presence of oxygen, which is introduced into the medium during cultivation (EP 0 885 962 B1):
   (2) 2 L-cysteine+½ O₂->L-cystine+H₂O
   Since the solubility of L-cystine in aqueous solution at a neutral pH is very low compared to cysteine, the disulfide already precipitates at a low concentration and forms a white precipitate:
   (3) L-cystine (dissolved)->L-cystine (precipitate)
   The precipitation of L-cystine lowers the level of the product dissolved in the medium, thereby also causing the reaction equilibrium of (1) and that of (2) to be pulled to the product side.
3) Purifying the product is significantly less technically complex if the amino acid can be obtained directly from the fermentation medium than if the product accumulates intracellularly and cell disruption is first required.

A cysteine production strain/microorganism strain capable of cysteine production/microorganism strain having a deregulated cysteine biosynthesis pathway/microorganism strain having deregulated cysteine biosynthesis is distinguished by at least one of the modifications/features selected from feedback-resistant SerA enzyme, feedback-resistant CysE enzyme and overexpression of a cysteine efflux protein.

Furthermore, it is known that the cysteine yield in fermentation can be increased by attenuating or destroying genes encoding cysteine-degrading enzymes, such as the tryptophanase TnaA or the cystathionine β-lyases MalY or MetC (EP 1 571 223 B1).

Besides the genetic modification of the cysteine production strain, the optimization of the fermentation process, i.e., how the cells are cultivated, also plays an important role in the development of an efficient production process. Various cultivation parameters, such as the nature and metering of the carbon and energy source, the temperature, the supply of oxygen (EP 2 707 492 B1), the pH and the composition of the culture medium, can have an influence on the product yield and/or the product spectrum in the fermentative production of cysteine.

Since raw material and energy costs are constantly rising, there is a constant need to increase the product yield in cysteine production so as to improve the economic viability of the process.

It is an object of the present invention to provide a microorganism strain for fermentative production of cysteine, L-cystine and/or thiazolidine, by means of which higher yields of L-cysteine, L-cystine and/or thiazolidine can be achieved in fermentation in comparison with known strains from the prior art.

The object is achieved by a microorganism strain comprising a deregulated cysteine biosynthesis pathway that is thereby suitable for fermentative production of at least one substance selected from L-cysteine, L-cystine and thiazolidine, characterized in that the relative expression of the crp gene is reduced in relation to the expression of the crp gene having a wild-type promoter sequence as a result of mutation of the crp promoter sequence.

Crp, which is encoded by the crp gene and is an abbreviation for cyclic AMP (cAMP) receptor protein (or for “catabolite repressor protein”) and is also known as CAP (catabolite activator protein), is a key transcription factor that is especially known for mediating so-called catabolite repression, i.e., regulating gene expression according to the carbon (C) source. For example, glucose is the preferred carbon source in E. coli and the expression of genes for metabolization of alternative carbon sources is suppressed (repressed) so long as glucose is available. Crp is activated by binding of the signaling molecule cAMP (cyclic AMP) (referred to as Crp-cAMP). As Crp-cAMP, it has an effect on the expression of target genes, thereby regulating not only the utilization of carbon sources, but also other cellular functions such as nitrogen fixation, biofilm formation, transport of the trace element iron or else osmotic equalization. Hanamura and Aiba, Nucleic Acids Res. (1991) 19:4413-4419 additionally report that Crp-cAMP can repress its own expression (negative autoregulation).

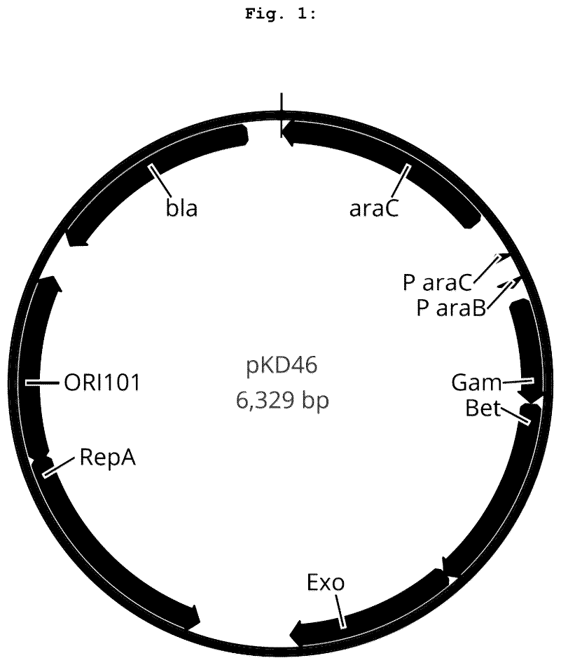

From transcriptome analyses (e.g., Gosset et al., J. Bacteriol. (2004) 186:3516-3524), it is known that the expression of a multitude of genes (>400 genes) is influenced by Crp, or Crp-cAMP. Furthermore, Crp is part of a branched regulatory network of global transcription factors which influence each other (outlined in FIG. 1 of Frendorf et al., Comput. Structural Biotechnol. J. (2019) 17:730-736) and which influence the expression of their target genes depending on the metabolic status of the cell.

If, then, the expression of the crp gene is altered, it is a priori not possible to state, because of the multitude of genes regulated by Crp-cAMP and because of the mutually influencing global transcription factors, how increased or attenuated crp expression will affect cellular metabolism generally or how it will affect a biosynthetic metabolic pathway, for example that for L-cysteine.

Frendorf et al. 2019 (see above) provide an overview of the multitude of hitherto studied mutants of the Crp protein. They are exclusively modifications of the Crp amino acid sequence, caused by modification of the crp cds. EP 3 686 214, EP 3 686 215 and EP 3 725 800 (all CJ Corp., Korea) likewise disclose mutants of the Crp amino acid sequence and the use thereof for producing L-amino acids, in particular L-threonine and L-tryptophan.

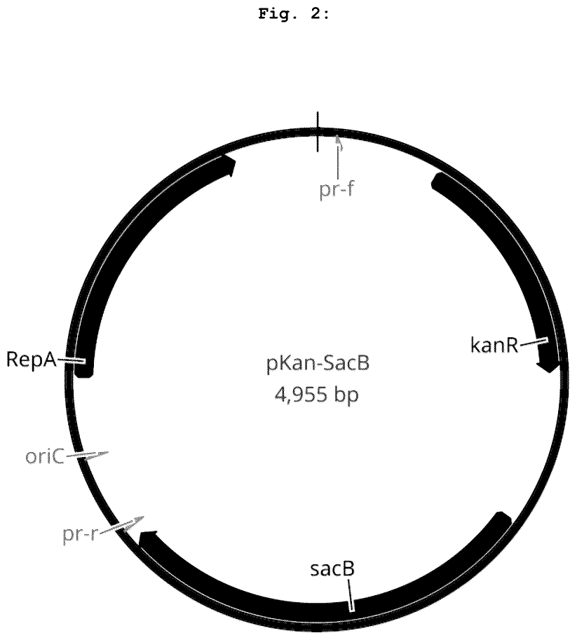

What has not been studied in the prior art is the effect on metabolism, in particular on cysteine biosynthesis, by altered expression of the crp WT gene due to a mutation in the crp promoter in a microorganism strain having a deregulated cysteine biosynthesis pathway. The present invention differs from the prior art in that it is preferably not the amino acid sequence of Crp that is altered, but the nucleotide sequence of the crp promoter. That is to say, what have been altered in the prior art are the amino acid sequence and hence the properties of the activity of the Crp protein as transcription factor. In contrast, the amino acid sequence and hence the properties of the activity of the Crp protein (wild-type Crp) preferably remain unaltered in the present invention; however, what is altered is expression, i.e., amount of protein, by modification of the crp promoter. From the prior art, no prediction could be made as to how this measure affects cellular metabolism generally and cysteine biosynthesis specifically in a microorganism strain having a deregulated cysteine biosynthesis pathway. For example, Liu et al., J. Agric. Food Chem 2020, 68:14928-14937, describe, in FIG. 2 of the publication, the E. coli strain BW25113-pLH03, which exhibited both increased crp gene expression and improved L-cysteine production compared to the strain JM109-pLH03. It was therefore completely unexpected that the reduced expression of the crp gene in a microorganism strain having a deregulated cysteine biosynthesis pathway, as disclosed in the present invention, leads to improvement in cysteine production.

The mutation of the crp promoter sequence leads to attenuated crp expression, preferably as a result of shortening of the crp promoter sequence or as a result of a combination of insertion and shortening of the crp promoter sequence, the crp cds particularly preferably remaining unaltered. The attenuated crp expression leads, in a hitherto unknown manner, to improved production of L-cysteine in a microorganism strain having a deregulated cysteine biosynthesis pathway. In a particularly preferred embodiment, the mutation of the crp promoter sequence leads to no Crp protein at all being expressed.

### Detection of Crp Gene Expression:

A method for quantitative detection of the expression of the crp gene is required so as to be able to compare the expression of the crp gene in various strains. Generally, various known test methods are available for quantitative detection of gene expression.
An immunological detection method involves binding of a specific antibody to the expressed Crp protein. In this way, the methods ELISA (“enzyme-linked immunosorbent assay”) and western blot that are known to a person skilled in the art allow the quantitative determination of the expressed protein by a color reaction which occurs via the bound Crp-specific antibody.
Another method of quantitative detection of gene expression involves determining the gene-specific RNA of an expressed gene such as crp RNA in the total cellular RNA (total RNA). In the context of the invention, the gene to be studied in respect of its expression is referred to as target gene (e.g., the crp gene). Methods known to a person skilled in the art are northern blot for direct detection of a gene-specific RNA and the preferred real-time PCR (RT-PCR), also known as qPCR (quantitative PCR), for indirect detection of a gene-specific RNA (e.g., crp RNA).
An RT-PCR analysis can be carried out, for example, as follows:
1) The cells to be analyzed (e.g., E. coli cells from cultivation (as described in Example 4) of the strains to be analyzed (Examples 3 and 5)) are admixed with an RNA-stabilizing reagent (e.g., RNA-Protect® “Bacteria Reagent” from Qiagen) after harvesting, and the total RNA is extracted (e.g., using the RNeasy RNA extraction kit from Qiagen) and quantified (e.g., by means of the “Qubit™ RNA BR Assay Kit” from Thermo Fisher Scientific).
2) The total RNA of the strains is used to produce complementary DNA (cDNA) by means of reverse transcription (e.g., using the QuantiNova™ Reverse Transcription Kit from Qiagen) and the cDNA is quantified (e.g., by means of the “Qubit™ dsDNA HS Assay Kit” from Thermo Fisher Scientific).
3) The cDNA is then, in accordance with the prior art, used in an RT-PCR reaction containing gene-specific primers for the target gene crp and gene-specific primers for a reference gene (e.g., the cysG gene). Reagent kits for preparing RT-PCR reactions (e.g., from Qiagen) and instruments for performing RT-PCR analyses, including evaluation software, are commercially available. For the analysis of crp expression described in Example 5, use was made of an RT-PCR instrument from Qiagen (RotorGene Q 2plex RT-PCR instrument, operated by the Rotor-Gene Q control and evaluation software from the same manufacturer), by means of which the relative expression of the crp gene in strains having a modified crp promoter was determined in relation to the comparative strain E. coli W3110 x pCys having a WT crp promoter.
   Relative expression: In the context of the invention, relative gene expression, determined as the 2^{-ΔΔCT} value, is defined as expression of the crp gene in the microorganism strain according to the invention having a deregulated cysteine biosynthesis pathway, in which the crp promoter sequence has been mutated (e.g., the cysteine production strains E. coli W3110-crp::kan-sacB x pCys, E. coli W3110-crpP-del x pCys, E. coli W3110-crp-Preg x pCys, E. coli W3110-crp-Preg2 x pCys and E. coli W3110-crp-Preg3 x pCys that are described in Example 3), in relation to the expression of the crp gene in a corresponding comparative strain (e.g., in the strain E. coli W3110 x pCys having a WT crp promoter). The RT-PCR analysis method used in Example 5 of the present invention involves the analysis of relative gene expression by means of quantitative RT-PCR and the so-called 2^{-ΔΔCT} method, as described by Livak and Schmittgen, Methods (2001) 25:402-408. This evaluation method forms the basis of the RotorGene Q control and evaluation software of the RotorGene Q 2plex RT-PCR instrument from Qiagen.
   CT value: The basis for determining relative gene expression is the so-called CT value (CT: “cycle threshold”), which, in the RT-PCR of the cDNA of a strain, is determined for both the crp gene (CT_{crp}) and a reference gene. The reference gene serves for normalization of the RT-PCR and is selected from known, constitutively expressed genes which are not subject to any regulation. In the context of the invention, the CT value for the cysG gene (CT_{cysG}) as reference gene was determined in Example 5, the expression of said gene being known to change only slightly over the course of cultivation (Zhou et al., BMC Molecular Biology (2011) 12:18).
   ΔCT value: In the context of the invention, the ΔCT value is defined as the difference between the CT values for the crp gene and the reference gene (e.g., cysG reference gene) of a strain. ΔCT=CT_{crp}−CT_{cycG}.
   ΔΔCT value: In the context of the invention, ΔΔCT is defined as the difference between the ΔCT value of a strain having altered expression of the crp gene and the ΔCT value of the comparative strain having WT expression of the target gene (e.g., E. coli W3110 x pCys). ΔΔCT=ΔCT-ΔCT_{W3110 x pCys}.
   2-ΔΔCT value: The value 2-ΔΔCT is formed from the ΔΔCT value and is a measure of the expression of the crp gene in a modified strain in comparison with expression of the crp gene in the comparative strain (e.g., E. coli W3110 x pCys), and is also referred to as relative expression of the target gene.
4) If the expression of the crp gene is to be compared between various strains, then the same amounts of cDNA are used in the RT-PCR and the relative crp expression as 2-ΔΔCT value is determined for each strain.
   The 2-ΔΔCT value for the comparative strain has a value of 1.
   2-ΔΔCT values>1 signify increased expression in comparison with the comparative strain.
   2-ΔΔCT values<1 signify attenuated expression in comparison with the comparative strain.
   From comparing the 2-ΔΔCT values for relative expression, it is then possible to state whether and the extent to which a mutation in the promoter sequence of the crp gene has altered the expression of the crp gene. This alteration of gene expression (cf. Example 5) can be correlated with altered properties of a strain, such as cell growth or yields of a metabolite, such as the production of the amino acid L-cysteine (cf. Examples 6 and 7).

Open reading frame (ORF, synonymous with cds or coding sequence) refers to that region of DNA or RNA that begins with a start codon and ends with a stop codon and encodes the amino acid sequence of a protein. The ORF is also referred to as the coding region or structural gene.

Gene refers to the section of DNA that contains all the basic information for producing a biologically active RNA. A gene contains the section of DNA from which a single-stranded RNA copy is produced by transcription and also the expression signals involved in the regulation of this copying process. The expression signals include for example at least one promoter, a transcription start, a translation start, and a ribosome binding site. In addition, a terminator and one or more operators are possible as expression signals.

Promoter refers to a nucleotide sequence which is upstream of the 5′ end of the cds and which allows the expression of a gene. The promoter comes before the RNA-coding region in the direction of synthesis. The promoter contains regions of specific interaction with DNA-binding proteins which mediate the start of transcription of the gene by the RNA polymerase and which are referred to as transcription factors.

Mutation refers to modification of genetic material, including modification of the DNA sequence and, if protein-coding sequences are concerned, also the amino acid sequence of proteins. In the context of the present invention, a mutation includes the exchange, insertion and/or deletion of one or more nucleotides or one or more amino acids. An exchange refers to the exchange of one or more nucleotides for other nucleotides in a DNA, or of one or more amino acids for other amino acids in a protein. At the same time, the length of the DNA sequence or the protein sequence remains unchanged. An insertion refers to the incorporation of additional nucleotides in a DNA, or of additional amino acids in a protein. The term insertion also covers elongations. In the case of a deletion, one or more nucleotides or else parts of a nucleotide sequence, or one or more amino acids or else parts of an amino acid sequence, are missing. The term point mutation is used if the modification results in only an individual nucleotide or an individual amino acid being exchanged for another nucleotide or another amino acid. Mutations also include the combination of exchange, deletion and insertion.

In the context of this invention, the proteins, for example Crp, start with a capital letter, whereas the gene of these protein with coding sequences are identified by a lowercase letter (e.g., crp).

Accordingly, the E. coli crp gene refers to—in SEQ ID NO: 1 from nucleotide 565-865—the promoter region of the crp gene and—SEQ ID NO: 1 from nucleotide 866-1495—the cds of the crp gene from E. coli. E. coli Crp refers to the protein encoded by said cds and specified in SEQ ID NO: 2. The protein is the Crp protein.

The abbreviation WT (Wt) refers to the wild type. Wild-type gene or wild-type promoter refers to the form of the gene or promoter that arose naturally through evolution and is present in the wild-type genome. The DNA sequence of Wt genes or Wt promoters is publicly available in databases such as NCBI (National Center for Biotechnology Information, U.S. National Library of Medicine).

Alleles define the states of a gene that can be converted into one another by mutation, i.e., by changes to the nucleotide sequence of the DNA. The gene that occurs naturally in a microorganism is referred to as the wild-type allele and the variants derived therefrom are referred to as the mutated alleles of the gene.

Homologous genes or homologous sequences are to be understood to mean that the DNA sequences of said genes or sections of DNA are at least 80% identical, preferably at least 90% identical and particularly preferably at least 95% identical.

Degree of DNA identity is determined by the “nucleotide blast” program which can be found at http://blast.ncbi.nlm.nih.gov/and which is based on the blastn algorithm. The algorithm parameters used to align two or more nucleotide sequences were the default parameters. The default general parameters are: Max target sequences=100; Short queries=“Automatically adjust parameters for short input sequences”; Expect Threshold=10; Word size=28; Automatically adjust parameters for short input sequences=0. The corresponding default scoring parameters are: Match/Mismatch Scores=1,−2; Gap Costs=Linear.

Protein sequences are compared using the “protein blast” program at http://blast.ncbi.nlm.nih.gov/. This program uses the blastp algorithm. The algorithm parameters used to align two or more protein sequences were the default parameters. The default general parameters are: Max target sequences=100; Short queries=“Automatically adjust parameters for short input sequences”; Expect Threshold=10; Word size=3; Automatically adjust parameters for short input sequences=0. The default scoring parameters are: Matrix=BLOSUM62; Gap Costs=Existence: 11 Extension: 1; Compositional adjustments=Conditional compositional score matrix adjustment.

In the case of the microorganisms according to the invention having a deregulated cysteine biosynthesis pathway, the mutation of the crp promoter sequence results in the relative expression of the crp gene being reduced preferably to a 2^{-ΔΔCT} value of at least 0.91, particularly preferably to a 2^{-ΔΔCT} value of at least 0.5 and especially preferably to a 2^{-ΔΔCT} value of 0.03, wherein the expression of the crp gene in microorganisms having a wild-type promoter is normalized to a 2^{-ΔΔCT} value of 1.00. The relative expression of the crp gene is preferably determined as described in Example 5.

If the expression of the crp gene having a wild-type promoter with a 2^{-ΔΔCT} value of 1.00 is defined as 100% gene expression of the crp gene, then crp gene expression in the microorganisms according to the invention with a 2^{-ΔΔCT} value reduced to at least 0.91, with a 2^{-ΔΔCT} value reduced to at least 0.5 and with a 2^{-ΔΔCT} value reduced to at least 0.03 is a maximum of 91%, a maximum of 50% and a maximum of 3%, respectively, of the expression of the crp gene having a wild-type promoter. That is to say, the process is preferably characterized in that crp expression in the microorganism strain having a modified crp promoter sequence is reduced by at least 9% in comparison with a corresponding microorganism strain having a Wt crp promoter sequence and the yield of L-cysteine in g/l is increased by at least 10% (w/v) when using the microorganism strain.

In the context of this invention, “compared to the/in comparison with the/in relation to the (corresponding) expression of the crp gene having a wild-type promoter (activity of the crp wild-type promoter, or WT expression)” means in comparison with the activity of the crp promoter that corresponds to the nonmutated form of the crp promoter from a microorganism, i.e., of the crp promoter which arose naturally through evolution and is present in the wild-type genome of said microorganism.

The microorganism strains suitable for fermentative production of L-cysteine, L-cystine or thiazolidine include all microorganisms which contain a deregulated cysteine biosynthesis pathway which leads to the synthesis of cysteine, cystine or thiazolidine. Such strains are disclosed, for example, in EP 0 885 962 B1, EP 1 382 684 B1, EP 1 220 940 B2, EP 1 769 080 B1 and EP 2 138 585 B1 and WO 2021/259491.

The microorganism strain having a deregulated cysteine biosynthesis pathway is distinguished by at least one of the following modifications:
a) The microorganism strain is distinguished by a modified serA gene encoding a 3-phosphoglycerate dehydrogenase (SerA) having a feedback inhibition by L-serine that is reduced by a factor of at least two in comparison with the corresponding wild-type enzyme (as described, for example, in EP 1 950 287 B1), wherein SerA enzyme activity can be determined photometrically by the oxidation of NADH dependent on the SerA substrate 3-phosphohydroxypyruvate, as described, for example, by McKitrick and Pizer, J. Bacteriol. (1980) 141:235-245.
   In comparison with the corresponding wild-type enzyme, particularly preferred variants of the 3-phosphoglycerate dehydrogenase (serA) have a feedback inhibition by L-serine that is reduced by a factor of at least 5, especially preferably by a factor of at least 10 and, in a more preferred embodiment, by a factor of at least 50.
   and/or
b) The microorganism strain contains a modified cysE gene encoding a serine O-acetyltransferase (CysE) which, in comparison with the corresponding wild-type enzyme, has a feedback inhibition by cysteine that is reduced by a factor of at least two (as described, for example, in EP 0 858 510 B1 or Nakamori et al. 1998 (see above)), wherein CysE enzyme activity can be determined photometrically by the consumption of the CysE substrate acetyl-CoA due to the reaction with L-serine to form O-acetyl-L-serine, as described, for example, by Nakamori et al. 1998 (see above).
   In comparison with the corresponding wild-type enzyme, particularly preferred variants of the serine O-acetyltransferase (cysE) have a feedback inhibition by cysteine that is reduced by a factor of at least 5, especially preferably by a factor of at least 10 and, in a more preferred embodiment, by a factor of at least 50.
   and/or
c) The microorganism strain exhibits, in comparison with the corresponding wild-type cell, an export of cysteine out of the cell that is increased by a factor of at least two owing to overexpression of an efflux gene, wherein the export of cysteine can be determined by photometric measurement of the extracellular content of cysteine according to the method by Gaitonde, Biochem. J. (1967) 104:627-633 (comprising cysteine, cystine and the adduct 2-methylthiazolidine-2,4 (R)-dicarboxylic acid formed from cysteine and pyruvate), as described, for example, in EP 0 885 962 B1.
   In comparison with a wild-type cell, the overexpression of an efflux gene preferably leads to an export of cysteine out of the cell that is increased by a factor of at least 5, particularly preferably by a factor of at least 10 and especially preferably by a factor of at least 20.
   The efflux gene preferably comes from the group consisting of ydeD (see EP 0 885 962 B1), yfik (see EP 1 382 684 B1), cydDC (see WO 2004/113373 A1), bcr (see US 2005-221453 AA) and emrAB (see US 2005-221453 AA) from E. coli or the correspondingly homologous gene from a different microorganism.
   and/or
d) The microorganism strain can furthermore be characterized in that at least one cysteine-degrading enzyme is attenuated to the extent that the cell contains only a maximum of 50% of this enzyme activity in comparison with a wild-type cell. The cysteine-degrading enzyme preferably comes from the group consisting of tryptophanase (TnaA) and cystathionine β-lyase (MalY, MetC).

Such strains are known, for example, from EP 0 858 510 B1 and EP 0 885 962 B1.

The microorganism strains suitable for fermentative production of L-cysteine, L-cystine or thiazolidine that are described in the previous paragraphs are deregulated in respect of their cysteine metabolism in such a way that they form an increased amount of L-cysteine in comparison with the microorganism strain which is not deregulated in respect of cysteine metabolism. This means that a cysteine production strain, or microorganism strain capable of cysteine production, is characterized in that it has a deregulated cysteine biosynthesis pathway. Since, in the cells of a microorganism strain which is not deregulated in respect of cysteine metabolism, the amount of L-cysteine in the culture is approximately 0 g/l, an increased amount preferably means any amount exceeding 0.05 g/l L-cysteine measured in the culture after 24 hours of cultivation.

The amount of cysteine can be quantified, for example, with the aid of the colorimetric assay by Gaitonde 1967 (see above), as described in Example 6 (cf. Table 2, strain W3110 and strain W3110 x pCys).

The microorganism strain according to the invention having a deregulated cysteine biosynthesis pathway, mutated crp promoter sequence and consequently reduced relative expression of the crp gene forms, in comparison with the corresponding microorganism strain—i.e., it is also distinguished by a deregulated cysteine biosynthesis pathway—with expression of the crp gene having a wild-type promoter, an increased amount of a substance selected from L-cysteine, L-cystine and thiazolidine, which is a great advantage. As demonstrated in Table 3 (Example 7), increasing mutation of the crp promoter sequence right up to complete deletion of the crp promoter, i.e., relative expression of the crp gene reduced by 9% to 100%, leads to significantly higher yields of total cysteine, i.e., the sum total of the cysteine, cystine and thiazolidine produced, in a fermentative process. A microorganism strain having a mutated promoter sequence in the crp gene is always compared with the corresponding microorganism strain having a Wt promoter sequence in the crp gene, i.e., both microorganism strains are deregulated in respect of their cysteine metabolism.

When cysteine is mentioned in the present invention, this always means L-cysteine or one of its derivatives selected from L-cystine or thiazolidine. This means that the amount of cysteine produced preferably means total cysteine, i.e., the sum total of the L-cysteine, L-cystine and thiazolidine produced. For example, L-cystine that has been formed can be reduced to form L-cysteine and then codetected by the measurement on cysteine produced. If determination is accomplished by using, for example, the colorimetric assay by Gaitonde (see above), said assay cannot distinguish between L-cysteine and the condensation product of cysteine and pyruvate, 2-methylthiazolidine-2,4-dicarboxylic acid (thiazolidine), described in EP 0 885 962 B1 under the highly acidic reaction conditions, and thiazolidine will also be codetected by the measurement on cysteine produced. According to the invention, the microorganism strain forms an increased amount of a substance selected from L-cysteine, L-cystine and thiazolidine, preferably L-cysteine and L-cystine and particularly preferably L-cysteine.

Preferably, the microorganism strain is characterized in that the amino acid sequence of the Crp protein is nonmutated. That is to say, the amino acid sequence of the Crp protein is the Wt sequence specified in SEQ ID NO: 2. In order to achieve this, the coding sequence of crp only comprises so-called silent mutations or no mutation at all. Silent mutations are due to the degeneracy of the genetic code and are defined as modifications of a cds that do not alter the amino acid sequence derived therefrom. This means that the crp cds is identical to the Wt DNA sequence available in the NCBI database for crp of the corresponding organism or only comprises silent mutations and encodes a nonmutated Crp protein having the Wt protein sequence. Particularly preferably in the present invention, it is only the promoter sequence of the crp gene of a microorganism strain that is mutated. Preference is given to mutations of the promoter sequence of the crp gene from E. coli, comprising the sequence SEQ ID NO: 1, nt 565-865, whereas the crp cds, comprising the sequence SEQ ID NO: 1, nt 866-1498, is nonmutated or only comprises silent mutations and encodes a protein having the protein sequence SEQ ID NO: 2.

In the context of the invention, mutations in the crp promoter sequence include modifications such
a) that the crp promoter sequence of the crp gene is partly or completely deleted and/or
b) that the crp promoter sequence of the crp gene is modified by one or more insertions or 5′ and/or 3′ elongations and/or
c) that the crp promoter sequence of the crp gene contains one or more point mutations,
with the result that the expression of the crp gene is reduced, i.e., attenuated or completely suppressed. As already described above, what is meant by reduced is that crp gene expression in the microorganisms according to the invention is preferably a maximum of 91%, particularly preferably a maximum of 50% and especially preferably a maximum of 3% of the expression of the crp gene having a wild-type promoter. In a specially preferred embodiment, there is no detectable crp gene expression in the microorganisms according to the invention.

Particularly preferably, the Crp protein does not have a mutation in the aforementioned cases of reduced crp gene expression, i.e., the amino acid sequence is unaltered in comparison with the Wt.

In the context of the invention, any desired combination of the genetic modifications in the promoter of the crp gene that are listed in a) to c) is also possible. In sum, in the context of the invention, the expression of the crp gene is attenuated or completely suppressed by modifications of the crp promoter. Attenuation of crp expression can also be achieved by replacing the crp promoter in full or in part by an alternative, weak promoter.

Particularly preferably, the modification of the crp promoter in the strain according to the invention involves complete or partial deletion of the crp promoter or modification of the crp promoter by one or more insertions or 5′ and/or 3′ elongations, or a combination of deletion and insertion.

Especially preferably, the modification of the crp promoter in the strain according to the invention involves complete or partial deletion of the crp promoter.

Preferably, the microorganism strain is characterized in that the mutation in the crp promoter sequence comprises at least one deletion or insertion, particularly preferably at least one deletion.

In a particularly preferred embodiment, the mutation in the crp promoter sequence, this preferably comprising the sequence specified in SEQ ID NO: 1 nt 565-865, is at least one deletion or insertion and the crp coding sequence is nonmutated.

Preferably, the microorganism strain is characterized in that at least nt 565-624 is deleted from the crp promoter sequence specified in SEQ ID NO: 1 nt 565-865. In this case, the crp promoter sequence comprises a maximum of nt 625-865 from SEQ ID NO: 1.

Especially preferably, the microorganism strain is characterized in that the crp promoter sequence specified in SEQ ID NO: 1 nt 565-865 is completely deleted.

Particularly preferred embodiments comprising insertion, deletion and elongation are shown in the examples:
for complete or partial deletion of the crp promoter sequence, see Example 2,
I for modification of the crp promoter sequence by one or more insertions, see Example 1,
for modification of the crp promoter sequence in a way that leads to weakening or complete inhibition of promoter activity, see Example 5.

Preferably, the microorganism strain is characterized in that the microorganism strain is a strain from the Enterobacteriaceae or Corynebacteriaceae family, particularly preferably a strain from the Enterobacteriaceae family. Such strains are commercially available from, inter alia, the DSMZ-German Collection of Microorganisms and Cell Cultures GmbH (Braunschweig) for example.

Preferably, the microorganism strain is selected from the group consisting of Escherichia coli, Pantoea ananatis and Corynebacterium glutamicum, particularly preferably from the group consisting of Escherichia coli and Pantoea ananatis. Especially preferably, the microorganism strain is a strain of the species Escherichia coli. Preferably, the E. coli strain is selected from E. coli K12, particularly preferably E. coli K12 W3110. Such strains are commercially available from, inter alia, the DSMZ-German Collection of Microorganisms and Cell Cultures GmbH (Braunschweig) for example, including E. coli K12 W3110 DSM 5911 (id. ATCC 27325) and Pantoea ananatis DSM 30070 (id. ATCC 11530).

The crp gene from E. coli K12 is, for example, accessible in the NCBI gene database as an entry in the E. coli Genbank Reference Sequence having accession number NC_000913.3, nt 3485255-nt 3486950 (SEQ ID NO: 1). The crp gene from Pantoea ananatis is, for example, accessible in the NCBI gene database as an entry in the P. ananatis Genbank Reference Sequence having accession number NC_017554.1, nt 430825-nt 431818 (crp cds: nt 430883-nt 431515; gene identification number 57266449).

In a preferred embodiment, the microorganism strain is characterized in that the mutated crp promoter sequence is selected from the group consisting of the promoter sequences of the crp gene from Escherichia coli and of the crp gene from Pantoea ananatis, and a homologous sequence in relation to these sequences, the term homologous sequence having the definition as specified above.

The crp gene is preferably the crp gene from E. coli having the promoter region specified in SEQ ID NO: 1 nt 565-865 and the crp cds specified in SEQ ID NO: 1, nt 866-1495, encoding a Crp protein having the amino acid sequence specified in SEQ ID NO: 2.

Accordingly, the microorganism strain is preferably characterized in that the expressed Crp protein is SEQ ID NO: 2. This means that the Crp protein which is expressed has the Wt sequence (SEQ ID No:2), and the mutation only concerns the crp promoter sequence (SEQ ID NO: 1 nt 565-865).

Furthermore, the production strain according to the invention can be yet further optimized for yet further improvement of cysteine production. Optimization can be achieved, for example, genetically by additionally expressing one or more genes suitable for improving production properties. Said genes can be expressed in the production strain in a manner known per se either as separate gene constructs or in combination as an expression unit (as a so-called operon). Furthermore, the production strain can be optimized by inactivating yet further genes, the gene products of which have an adverse effect on cysteine production, in addition to the reduction of the expression of the crp gene. However, optimization is also possible in a manner known per se by mutagenesis and selection of strains having improved cysteine production.

In an alternative approach, it is also conceivable to attenuate or completely suppress the expression of the crp gene by adding an inhibitor, be it a chemical inhibitor or protein inhibitor, said inhibitor having an inhibitory effect on the activity of the crp promoter and not on the activity of the Crp protein.

Various methods for introducing modifications in the promoter of the crp gene are known to a person skilled in the art. In the simplest case, the parent strain can be subjected to mutagenesis in a known manner (e.g., chemically by means of mutagenic chemicals such as N-methyl-N′-nitro-N-nitrosoguanidine or physically by means of UV irradiation), with mutations being randomly generated in the genomic DNA and the desired mutant having a modified crp promoter then being selected from the multitude of mutants generated, for example after the mutants have each been singularized, by quantitative determination of the crp protein (e.g., immunologically by western blot using a crp-specific antibody) or by quantitative determination of crp expression by means of, for example, RT-PCR. Only mutants having a modified crp promoter are selected in each case, while the crp cds remains unaltered and corresponds to the wild-type sequence.

In contrast to complex random mutagenesis and selection of the sought-after mutant having a modified crp promoter, the promoter of the crp gene can be subjected to targeted modification in a relatively simple manner, for example by the known mechanism of homologous recombination. Cloning systems for targeted gene inactivation by means of homologous recombination are known to a person skilled in the art and commercially available, as disclosed, for example, in the user manual of the “Quick and Easy E. coli Gene Deletion Kit”, based on Red®/ET® technology from Gene Bridges GmbH (see “Technical Protocol, Quick & Easy E. coli Gene Deletion Kit, by Red®/ET® Recombination, Cat. No. K006, Version 2.3, June 2012” and the references cited therein).

According to the prior art, the crp promoter or part of the promoter can be isolated and foreign DNA can be cloned into the crp promoter, thereby altering the sequence of the promoter. A DNA construct suitable for the targeted modification of the crp promoter can thus consist of a 5′ section of DNA which is homologous to the genomic crp promoter, followed by a gene segment comprising the foreign DNA, followed by a 3′ section of DNA which is again homologous to the genomic crp promoter.

The possible region in the crp promoter for homologous recombination can comprise not just the promoter sequence region. The possible region can also comprise DNA sequences which flank the promoter, namely the 5′-flanking sequence before the start of the crp promoter (e.g., nt 1-564 in SEQ ID NO: 1). DNA sequences in the 3′ region of the crp promoter concern the cds of the crp gene (crp cds, nt 866-1498 in SEQ ID NO: 1), in which case modification of the cds of the crp gene by homologous recombination is excluded. The foreign DNA is preferably a selection-marker expression cassette, for example selected from the class of antibiotic resistance genes.

Another such system for targeted gene inactivation based on homologous recombination is a method for gene modification that is based on a combination of Lambda Red recombination with counter-selection screening and that is known to a person skilled in the art and described in Examples 1 and 2. Said system is described, for example, in Sun et al., Appl. Env. Microbiol. (2008) 74:4241-4245. Use is made of a DNA construct for inactivating, for example, the crp promoter, consisting, starting from the 5′ end, of a sequence homologous to the crp gene (comprising the 5′ region of the crp promoter) followed by two expression cassettes in any order, consisting of a) an expression cassette of the selection marker selected from the class of the antibiotic resistance genes and b) an expression cassette of the sacB gene encoding the enzyme levansucrase, and lastly followed by a further sequence homologous to the crp gene (comprising, for example, sequences of the crp cds which 3′-flank the crp promoter).

In a first step, the DNA construct is transformed into the production strain and antibiotic-resistant clones are isolated. The clones obtained are distinguished by the fact that they cannot grow on sucrose owing to the coincorporated sacB gene. The two marker genes can be removed by the principle of counter-selection, in that, in a second step, a suitable DNA fragment replaces the two marker genes by homologous recombination. The clones obtained in this step then regain their ability to grow on sucrose and then also regain their sensitivity to the antibiotic. This method is used in Examples 1 and 2 for targeted shortening of the crp promoter of E. coli (SEQ ID NO: 1, nt 565-865). A DNA fragment suitable for this step comprises, starting from the 5′ end, a sequence of at least 20 nt in length that is homologous to the target gene, for example the crp gene, followed by a section of DNA that contains the desired altered DNA sequence, for example a shortened crp promoter, and lastly a further sequence of at least 20 nt in length that is homologous to the target gene, for example the crp gene. The DNA fragment can be, for example, produced chemically by gene synthesis or as in Example 2, from individual DNA fragments by the known and so-called OE-PCR (Overlap Extension PCR, as described, for example, in Hilgarth and Lanigan, MethodsX (2020) 7:100759, https://doi.org/10.1016/j.mex.2019.12.001).

The following E. coli strain is disclosed in the examples as an example of a strain according to the invention that has attenuated crp expression owing to a combination of insertion of the Kan-sacB cassette and deletion in the crp promoter: W3110-crp::kan-sacB. In W3110-crp::kan-sacB, the 3.2 kb Kan-sacB cassette is inserted in the crp promoter between nt 640 and nt 714 of SEQ ID NO: 1, as a result of which 73 nt (SEQ ID NO: 1, nt 641-nt 713) of the crp promoter has been deleted at the same time (see Example 1). As a consequence of this modification of the crp promoter, the relative expression of the crp gene in the strain W3110-crp::kan-sacB x pCys transformed with the plasmid pCys was only 0.33 times, i.e., only 33% of, the expression in the strain W3110 x pCys having a wild-type crp promoter normalized to 1 or 100% (see Example 5, Table 1).

The following E. coli strains are disclosed in the examples (Example 2) as examples of strains according to the invention that have attenuated crp expression owing to shortening of the crp promoter:
W3110-crpP-del: deletion of nt 565-nt 865 from SEQ ID NO: 1
W3110-crp-Preg: deletion of nt 565-nt 713 from SEQ ID NO: 1
W3110-crp-Preg2: deletion of nt 565-nt 675 from SEQ ID NO: 1
W3110-crp-Preg3: deletion of nt 565-nt 624 from SEQ ID NO: 1

As a consequence of these instances of shortening the crp promoter, the relative expression of the crp gene in the strains transformed with the plasmid pCys was only the following fractions of the expression in the strain W3110 x pCys having a wild-type crp promoter normalized to 1 (see Example 5, Table 1): In E. coli W3110-crpP-del x pCys having a completely deleted crp promoter (deletion of nt 565-nt 865 from SEQ ID NO: 1), relative crp expression was only 0.03 times that for the wild-type crp promoter. In E. coli W3110-crp-Preg x pCys, the crp promoter had been shortened by 149 nt (deletion of nt 565-nt 713 from SEQ ID NO: 1). Relative crp expression was only 0.05 times that for the wild-type crp promoter. In E. coli W3110-crp-Preg2 x pCys, the crp promoter had been shortened by 111 nt (deletion of nt 565-nt 675 from SEQ ID NO: 1). Relative crp expression was only 0.5 times that for the wild-type crp promoter. In E. coli W3110-crp-Preg3 x pCys, the crp promoter had been shortened by 60 nt (deletion of nt 565-nt 624 from SEQ ID NO: 1). Relative crp expression was only 0.91 times that for the wild-type crp promoter.

These results show that, on the one hand, increasing shortening of the crp promoter reduced the relative expression of the crp gene to from 0.91 times to 0.03 times the expression for the Wt promotor or that a combination of insertion and deletion attenuated the relative expression of the crp gene to 0.33 times the expression for the Wt promotor.

For the E. coli crp gene, it is preferred to attenuate the relative expression of the crp gene from a value of 1 for the wild-type crp promoter to at least 0.91 times, particularly preferably to at least 0.5 times and especially preferably to at least 0.33 times said value by deletion or a combination of insertion and deletion.

The strain according to the invention, characterized by modification of the crp promoter in a way that leads to attenuation of crp expression, such as the E. coli strains W3110-crp::kan-sacB, W3110-crpP-del, W3110-crp-Preg, W3110-crp-Preg2 or W3110-crp-Preg3, can be produced by using the above-described combination of Lambda Red recombination with counter-selection screening for genetic modification (see, for example, Sun et al. 2008, see above), as disclosed in Examples 1 and 2.

Particularly preferred strains are E. coli W3110-crp-Preg2 and E. coli W3110-crp-Preg3 (described in Example 2).

The invention further provides a process comprising the production of at least one compound selected from L-cysteine, L-cystine and thiazolidine, characterized in that the microorganism strains according to the invention are used. The process can comprise cultivation of the microorganism strains according to the invention in a shake flask (laboratory scale) or fermenter (production scale), preference being given to a process in a fermenter (production scale). Although a shake flask culture also involves specification of a specific medium and pH and cultivation in the presence of oxygen and under constant motion (shaking), it is possible in a fermenter to set and regulate more defined conditions concerning the medium (e.g., supply of components or regulation of fermenter volume by partial draining of fermenter broth), temperature, pH, supply of oxygen and mixing of medium. That is to say, both a shake flask culture and a fermenter culture are referred to as fermentative processes and differ in scale. In a fermentative process, the microbial production strain is made to grow and produce the metabolite. A culture on a smaller scale can also be used as a preculture for inoculation of a culture on a larger scale.

The prior art does not disclose processes or production strains in which attenuation of crp expression by modification of the crp promoter sequence can improve the production of cysteine.

The primary product of the process according to the invention that is formed is L-cysteine. Oxidation yields sparingly soluble L-cystine according to equations (1) to (3), which accumulates as precipitate during fermentation (EP 0 885 962 B1, EP 2 707 492 B1). Formation of an adduct with pyruvate yields thiazolidine, which accumulates in the culture supernatant (EP 0 885 962 B1).

Preferably, the process is characterized in that the L-cysteine, L-cystine or thiazolidine formed is isolated. The isolation of L-cysteine is disclosed in EP 2 699 544 B1 and EP 1 958 933 B1. Precipitated L-cystine can be removed from the rest of the constituents, for example with the aid of a decanter, followed by dissolution of the crude product with a mineral acid, clarification of the crude product solution by centrifugation or filtration, decoloring of the solution and precipitation crystallization (EP 2 707 492 B1).

In the context of this invention, the yield of total cysteine is defined as the sum total of the cysteine, cystine and thiazolidine produced. This is determined from the entire culture, as described in Example 7. It can be quantified, for example, with the aid of the colorimetric assay by Gaitonde (Gaitonde, M. K. (1967), Biochem. J. 104, 627-633).

As shown in the examples of the present application, the attenuation of crp expression in a microorganism strain having deregulated cysteine biosynthesis that is suitable for cysteine, cystine or thiazolidine production is suitable for significantly increasing the yields of total cysteine, i.e., the sum total of the cysteine, cystine and thiazolidine produced, in a fermentative process. From the prior art, this was completely unexpected.

It is surprising that the fermentation of microorganism strains having a deregulated cysteine biosynthesis pathway and having reduced expression of the crp gene due to mutation of the crp promoter sequence in relation to the expression of the crp gene having a wild-type promoter sequence leads to significantly higher cysteine yields. The evidence summarized in Table 3 of Example 7 shows that fermentation of the strains E. coli W3110-crp::kan-sacB x pCys with 0.33 times relative expression of the crp gene, W3110-crp-Preg2 x pCys with 0.5 times relative expression of the crp gene and W3110-crp-Preg3 x pCys with 0.91 times relative expression of the crp gene achieved significantly higher cysteine yields in comparison with the corresponding wild-type strain W3110 x pCys with relative crp expression of 1. Contrary to the prior art and unexpectedly for a person skilled in the art, the attenuation of expression of the crp gene by complete or partial deletion, or a combination of insertion and deletion, in the crp promoter led to improved cysteine-producing strains.

This novel and inventive measure for improving cysteine-producing strains was confirmed by the results summarized in Table 2 of Example 6, in which the attenuation of expression of the crp gene by deletion, or a combination of insertion and deletion, in the crp promoter already led to improved cysteine yields in cultivation in shake flasks. Furthermore, Table 2 of Example 6 illustrates that there was also impairment of cell growth (OD600/ml in Table 2) in the case of strains having greatly attenuated crp expression, such as W3110-crpP-del x pCys (0.03-times relative crp expression compared to W3110 x pCys) and W3110-crp-Preg x pCys (0.05 times relative expression compared to W3110 x pCys). Nevertheless, there was a distinct improvement in cysteine production, which was even more pronounced when cysteine production was based on growth (mg of cysteine/OD in Table 2), corresponding to an increase by 285.5% or by 360%.

For a person skilled in the art, the attenuation of expression of the crp gene by deletion, or a combination of insertion and deletion, in the crp promoter is therefore a novel useful measure for improving cysteine production in other cysteine-producing strains as well. Accordingly, in the microorganism strain according to the invention that is suitable for cysteine production and has deregulated cysteine biosynthesis, the expression of the crp gene is attenuated by deletion, or a combination of insertion and deletion, in the crp promoter and cysteine production is increased at the same time, there being exclusion of modification of the crp cds.

Example 7 demonstrates that a strain capable of cysteine production and having deregulated cysteine biosynthesis and having attenuated expression of the crp gene by deletion, or a combination of insertion and deletion, in the crp promoter achieves significantly higher cysteine yields in fermentation than a strain containing the WT promoter of the crp gene, there being no alteration of the crp cds in all the strains according to the invention.

In the fermentative process in question, what are formed are not only biomass of the production strain according to the invention, but also cysteine and its oxidation product cystine. The formation of biomass and cysteine can temporally correlate, or biomass and cysteine can be formed over time in a mutually decoupled manner. Cultivation is carried out in a manner familiar to a person skilled in the art. To this end, cultivation can be carried out in shake flasks (laboratory scale) or else in a fermenter (production scale).

The microorganism strain is characterized in that it is deregulated in respect of the cysteine biosynthesis pathway and contains at least one mutation in the promoter of the crp gene. At the same time, the strain forms an increased amount of L-cysteine in comparison with the strain having a wild-type crp promoter. Preferably, the genetic modification in the promoter of the crp gene leads to reduction of the expression of the crp gene by at least 9% (2^{-ΔΔCT} value≤0.91), particularly preferably by at least 50% (2^{-ΔΔCT} value≤0.5) and especially preferably by at least 67% (2^{-ΔΔCT} value≤0.33) compared to the wild-type crp promoter (100% expression, 2^{-ΔΔCT} value=1).

Preferably, the process is characterized in that crp expression in the microorganism strain having a modified crp promoter sequence is reduced by at least 9%, particularly preferably by at least 50% and especially preferably by at least 67% in comparison with a corresponding microorganism strain having a Wt crp promoter sequence and the yield of a substance selected from L-cysteine, L-cystine and thiazolidine in g/l is increased by at least 10% (w/v), particularly preferably by at least 20% (w/v) and especially preferably by at least 50% (w/v) when using the microorganism strain. According to the invention, the microorganism strain forms an increased amount of a substance selected from L-cysteine, L-cystine and thiazolidine, preferably L-cysteine and L-cystine and particularly preferably L-cysteine.

As a result of the reduced crp expression, total cysteine production (amount produced per volume in g/L), i.e., of the cysteine, cystine and thiazolidine produced, in the case of cultivation in a shake flask or in a fermenter is increased preferably by at least 10% (w/v), particularly preferably by at least 20% (w/v) and especially preferably by at least 50% (w/v) compared to the comparative strain having a WT crp promoter. The amount produced per volume in shake flask cultivation within 24 h is preferably at least 0.37 g/L (Table 2) and the amount produced per volume in fermentation within 48 h is preferably at least 15.9 g/L (Table 3).

Preferably, the process is characterized in that the process is a fermentative process and the fermentation volume is at least 1 L, particularly preferably greater than 10 L, especially preferably greater than 1000 L and specifically preferably greater than 10000 L. Particularly preferably, the fermentative process is a process in a fermenter.

Cultivation media are familiar to a person skilled in the art from the practice of microbial cultivation. They typically consist of a carbon source, a nitrogen source, and additives such as vitamins, salts and trace elements, and a sulfur source, by means of which cell growth and cysteine production are optimized.

Carbon sources are those that can be used by the production strain for formation of cysteine product. These include all forms of monosaccharides, including C6 sugars (hexoses) such as glucose, mannose, fructose or galactose and C5 sugars (pentoses) such as xylose, arabinose or ribose, and all conceivable di- and polysaccharides formed therefrom, such as sucrose, lactose, maltose, maltodextrin, starch, and the monomers or oligomers (enzymatically or chemically) released therefrom by hydrolysis. Other usable carbon sources other than sugars or carbohydrates are acetic acid (and acetate salts derived therefrom), ethanol, glycerol, citric acid (and salts thereof) or pyruvate (and salts thereof). However, gaseous carbon sources such as carbon dioxide or carbon monoxide are also conceivable.

Preferred carbon sources for cultivating the production strains are glucose, fructose, sucrose, mannose, xylose and arabinose, including particularly preferably glucose and sucrose and especially preferably glucose.

Nitrogen sources are those that can be used by the production strain for formation of biomass. These include ammonia, in gaseous form or in aqueous solution as NH₄OH, or else salts thereof such as ammonium sulfate, ammonium chloride, ammonium phosphate, ammonium acetate or ammonium nitrate. Furthermore, suitable nitrogen sources are the known nitrate salts such as KNO₃, NaNO₃, ammonium nitrate, Ca(NO₃)₂, Mg(NO₃)₂ and other nitrogen sources such as urea. The nitrogen sources also include complex mixtures of amino acids such as yeast extract, proteose peptone, malt extract, soy peptone, casamino acids, corn steep liquor (liquid or else dried as so-called CSD) and also NZ Amine and yeast nitrogen base.

The metered addition of a sulfur source, either as a one-time addition in batch form or as a continuous feed, is required for the efficient production of cysteine and cysteine derivatives. Continuous metered addition can be effected as a pure feed solution or else in a mixture with a further feed component such as glucose. Suitable sulfur sources are salts of sulfates, sulfites, dithionites, thiosulfates or sulfides, and it is also conceivable to use the respective acids at a given stability. Preferred sulfur sources are salts of sulfates, sulfites, thiosulfates and sulfides, including particularly preferably salts of sulfates and thiosulfates and especially preferably thiosulfate salts, such as sodium thiosulfate and ammonium thiosulfate.

Cultivation can be carried out in so-called batch mode, comprising inoculation of the cultivation medium with a starter culture of the production strain and then cell growth without further feeding of nutrient sources. Cultivation can also be carried out in so-called fed-batch mode, comprising additional feeding of nutrient sources (feed) after an initial phase of growth in batch mode in order to compensate for the consumption thereof. The feed can consist of the carbon source, the nitrogen source, the sulfur source, one or more vitamins or trace elements important for production, or a combination of the foregoing. The feed components can be metered in together as a mixture or else separately in individual feed sections. In addition, other media constituents can also be added to the feed, as can additives which specifically increase cysteine production. The feed can be supplied continuously or in portions (discontinuously), or else in a combination of continuous and discontinuous feed. Preference is given to cultivation in fed-batch mode.

Preferred carbon sources in the feed are glucose, sucrose, and glucose- or sucrose-containing vegetable hydrolysates, and mixtures of the preferred carbon sources in any mixing ratio. A particularly preferred carbon source in the feed is glucose.

Preferably, the carbon source of the culture is metered in such that the content of the carbon source in the fermenter during the production phase does not exceed 10 g/L. A maximum concentration of 2 g/L is preferred, that of 0.5 g/L is particularly preferred, and that of 0.1 g/L is especially preferred.

Preferred nitrogen sources in the feed are ammonia, in gaseous form or in aqueous solution as NH₄OH, and its salts ammonium sulfate, ammonium phosphate, ammonium acetate and ammonium chloride, and additionally urea, KNO₃, NaNO₃ and ammonium nitrate, yeast extract, proteose peptone, malt extract, soy peptone, casamino acids, corn steep liquor and also NZ Amine and yeast nitrogen base, including particularly preferably ammonia or ammonium salts, yeast extract, soy peptone or corn steep liquor (in liquid or in dried form).

Preferred sulfur sources in the feed are salts of sulfates, sulfites, thiosulfates and sulfides, including particularly preferably salts of sulfates and thiosulfates and especially preferably thiosulfate salts, such as sodium thiosulfate and ammonium thiosulfate.

As further media additives, salts of the elements phosphorus, chlorine, sodium, magnesium, nitrogen, potassium, calcium, iron and, in trace amounts (i.e., in μM concentrations), salts of the elements molybdenum, boron, cobalt, manganese, zinc, copper and nickel can be added. Furthermore, organic acids (e.g., acetate, citrate), amino acids (e.g., isoleucine) and vitamins (e.g., vitamin B1, vitamin B6) can be added to the medium.

Cultivation is carried out under pH and temperature conditions which promote growth and cysteine production of the production strain. The useful pH range ranges from pH 5 to pH 9. Preference is given to a pH range from pH 5.5 to pH 8. Particular preference is given to a pH range from pH 6.0 to pH 7.5.

The preferred temperature range for growth of the production strain is 20° C. to 40° C. The temperature range is particularly preferably from 25° C. to 37° C. and especially preferably from 28° C. to 34° C.

Growth of the production strain can optionally take place without supply of oxygen (anaerobic cultivation) or else with supply of oxygen (aerobic cultivation). Preference is given to aerobic cultivation with oxygen.

In the case of aerobic cultivation of the strain according to the invention for cysteine production, a saturation of at least 10% (v/v), preferably of at least 20% (v/v) and particularly preferably of at least 30% (v/v) is set for the oxygen content. In accordance with the prior art, the oxygen saturation in the culture is regulated automatically via a combination of gas supply and stirring speed.

The supply of oxygen is ensured by introduction of compressed air or pure oxygen. Preference is given to aerobic cultivation by introduction of compressed air. The useful range of the compressed air supply in aerobic cultivation is 0.05 vvm to 10 vvm (vvm: introduction of compressed air into the fermentation batch specified in liters of compressed air per liter of fermentation volume per minute). An introduction of compressed air of 0.2 vvm to 8 vvm is preferred, that of 0.4 to 6 vvm is particularly preferred, and that of 0.8 to 5 vvm is especially preferred.

The maximum stirring speed is 2500 rpm, preferably 2000 rpm and particularly preferably 1800 rpm.

The cultivation time is between 10 h and 200 h. Preference is given to a cultivation time of 20 h to 120 h. Particular preference is given to a cultivation time of 30 h to 100 h.

Cultivation batches obtained by the process described above contain L-cystine in precipitated form, which is formed from the primary product L-cysteine according to equations (1) to (3) (EP 0 885 962 B1, EP 2 707 492 B1). Depending on the fermentation conditions, L-cysteine can also be the main product, which accumulates in dissolved form in the culture supernatant (EP 2 726 625 B1). The cysteine or cystine contained in the cultivation batches can either be further used directly without further work-up or else be isolated from the cultivation batch.

Preferably, the process is characterized in that the cysteine or cystine formed is isolated. Process steps known per se are available for isolating the cysteine and cystine, including centrifugation, decantation, dissolution of the crude product with a mineral acid, filtration, extraction, chromatography or crystallization or precipitation. Said process steps can be combined in any form in order to isolate the cysteine in the desired purity. The desired degree of purity depends on the further use. Processes for isolating L-cysteine are disclosed in EP 2 699 544 B1 and EP 1 958 933 B1. The procedure for isolating L-cystine is described in EP 2 707 492 B1.

The cystine obtained by work-up can be reduced to cysteine for further use. A process for reducing L-cystine to L-cysteine in an electrochemical process is disclosed in EP 0 235 908.

Various analytical methods for identifying, quantifying and determining the degree of purity of the cysteine or cystine product are available, including spectrophotometry, NMR, gas chromatography, HPLC, mass spectroscopy, gravimetry or else a combination of these analytical methods.

The invention can also be used to produce improved microorganism strains for fermentative production of compounds, the biosynthesis of which starts from 3-phosphoglycerate and leads to L-cysteine and L-cystine via L-serine. This also includes microorganism strains for fermentative production of derivatives of L-serine and L-cysteine, including phosphoserine, O-acetylserine, N-acetylserine and thiazolidine.

The figures show the plasmids used in the examples.

FIG. 1 shows the 6.3 kb vector pKD46 used in Example 1 and Example 2.

FIG. 2 shows the 5 kb vector pKan-SacB used in Example 1.

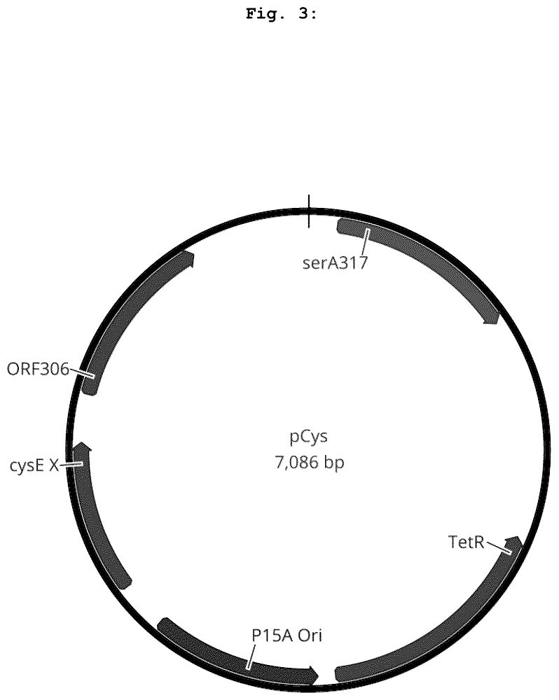

FIG. 3 shows the 7.1 kb vector pCys used in Example 3.

### Abbreviations Used in the Figures

bla: Gene conferring resistance to ampicillin (β-lactamase)
kanR: Gene conferring resistance to kanamycin
araC: araC gene (repressor gene)
ParaC: Promoter of the araC gene
ParaB: Promoter of the araB gene
Gam: Lambda phage Gam recombination gene
Bet: Lambda phage Bet recombination gene
Exo: Lambda phage Exo recombination gene
ORI101: Temperature-sensitive origin of replication
RepA: Gene for plasmid replication protein A
sacB: Levansucrase gene
pr-f: Binding site f for primer (forward)
pr-r: Binding site r for primer (reverse)
OriC: Origin of replication C
TetR: Gene conferring resistance to tetracycline
P15A ORI: Origin of replication
serA317: serA (3-phosphoglycerate dehydrogenase gene, encodes amino acids 1 to 317) cds
cysE X: cysE (serine O-acetyltransferase gene, feedback-resistant) cds
ORF306: ydeD (cysteine efflux gene) cds

The invention will be further illustrated by the following examples without being restricted by them:

### EXAMPLE 1: PRODUCTION OF THE STRAIN E. COLI W3110-CRP::KAN-SACB

The parent strain used for DNA isolation and for strain development was Escherichia coli K12 W3110 (commercially available under the strain number DSM 5911 from the DSMZ-German Collection of Microorganisms and Cell Cultures GmbH).

The target of gene modification was the promoter region of the crp gene from E. coli. The DNA sequence of the crp gene region, comprising the cds of the divergently expressed yhfA gene, the crp promoter region and the cds of the crp gene from E. coli K12 (Genbank NCBI Reference Sequence NC_000913.3, nt 3485255-nt 3486950), is disclosed in SEQ ID NO: 1.

Nucleotides 163-564 (identified by E. coli yhfA) comprise, in reverse-complementary form, the cds of the yhfA gene.

Nucleotides 866-1495 (identified by E. coli crp) comprise the cds of the crp gene, encoding a protein having the amino acid sequence of SEQ ID NO: 2.

The intergenic region between the divergently expressed genes yhfA and crp, comprising nucleotides 565-865 in SEQ ID NO: 1, contains the promoter sequence of the crp gene. Analysis of the crp promoter region is described in Hanamura and Ajba 1991 (see above).

The strain E. coli W3110-crp::kan-sacB, characterized by integration of the kan-sacB cassette in the crp promoter, was produced by using the combination of Lambda Red recombination and counter-selection screening for genetic modification that is known to a person skilled in the art (see, for example, Sun et al. 2008; see above).

The procedure was as follows:
1. E. coli W3110 was transformed with the plasmid pKD46 and transformants were selected on LBamp plates (10 g/L tryptone from GIBCO™, 5 g/L yeast extract from BD Biosciences, 5 g/L NaCl, 1.5% agar, 100 mg/L ampicillin from Sigma-Aldrich). An ampicillin-resistant clone was selected and designated E. coli W3110 x pKD46. The 6.3 kb plasmid pKD46 (so-called “Red Recombinase” plasmid, FIG. 1) is disclosed in the “GenBank” gene database under the accession number AY048746.1.
2. The 3.2 kb Kan-sacB cassette was isolated from the plasmid pKan-SacB by PCR using the primers crp-9f (SEQ ID NO: 3) and crp-10r (SEQ ID NO: 4). The 5 kb plasmid pKan-sacB (FIG. 2) contains expression cassettes for both the kanamycin (kanR) resistance gene and the sacB gene encoding the enzyme levansucrase. The E. coli kanamycin resistance gene encoding an aminoglycoside phosphotransferase is disclosed in the NCBI database under the accession number SH02_03400. The B. subtilis sacB gene is disclosed in the NCBI database under the accession number 936413.
   The primer crp-9f contained 50 nt from the crp promoter region (nt 591-640 in SEQ ID NO: 1) and, connected thereto, 20 nt specific for the plasmid pKan-SacB (referred to as “pr-f” in FIG. 2). The primer crp-10r contained 51 nt from the crp promoter region (nt 714-764 in SEQ ID NO: 1, in reverse-complementary form) and, connected thereto, 21 nt specific for the plasmid pKan-SacB (referred to as “pr-r” in FIG. 2).
3. E. coli W3110 x pKD46 was transformed with the 3.2 kb PCR product specific for the crp promoter region and kanamycin-resistant clones were isolated on LBkan plates (10 g/L tryptone, 5 g/L yeast extract, 5 g/L NaCl, 1.5% agar, 15 mg/L kanamycin).
4. Kanamycin-resistant clones were seeded onto LBSC plates (10 g/L tryptone, 5 g/L yeast extract, 7% sucrose, 1.5% agar and 15 mg/L kanamycin). Clones having an integrated sacB gene produced toxic levan from the sucrose, which led to inhibition of growth.
5. From cells from cultivation of kanamycin-resistant and sucrose-sensitive clones in LBkan medium (10 g/L tryptone, 5 g/L yeast extract, 5 g/L NaCl, 15 mg/L kanamycin), genomic DNA was isolated using a DNA isolation kit (Qiagen).
6. Genomic DNA was used in a PCR reaction (“Phusion™ High-Fidelity” DNA polymerase, Thermo Scientific™) with the primers crp-7f (SEQ ID NO: 5) and crp-8r (SEQ ID NO: 6) to verify the integration of the Kan-sacB cassette.
   In the PCR reaction, E. coli W3110 wild-type DNA yielded a DNA fragment of 1696 nt (corresponding to the sequence from SEQ ID NO: 1), as expected for the intact gene structure composed of yhfA cds, crp promoter and crp cds. Kanamycin-resistant clones by contrast yielded in the PCR reaction a DNA fragment of approx. 4800 nt, as expected for integration of the 3.2 kb PCR product at the sites in the crp promoter defined by the primers crp-9f and crp-10r. As a result of integration of the 3.2 kb Kan-sacB cassette at the sites defined by the primers crp-9f and crp-10r, confirmed by DNA sequencing of the 4800 nt PCR product, 74 nucleotides were removed from the crp promoter (SEQ ID NO: 1, nt 641-nt 713).
7. A clone having an integrated Kan-sacB cassette was selected and designated W3110-crp::kan-sacB x pKD46.
8. The temperature-sensitive plasmid pKD46 was removed by incubation at 42° C. Clones without the pKD46 plasmid were ampicillin-sensitive and were no longer able to grow on LBamp plates. An ampicillin-sensitive clone was selected and designated W3110-crp::kan-sacB.

### EXAMPLE 2: PRODUCTION OF W3110 STRAINS HAVING A DIFFERENTLY SHORTENED CRP Promoter

The following W3110 strains having a shortened crp promoter were produced:
W3110-crpP-del: deletion of nt 565-nt 865 from SEQ ID NO: 1
W3110-crp-Preg: deletion of nt 565-nt 713 from SEQ ID NO: 1
W3110-crp-Preg2: deletion of nt 565-nt 675 from SEQ ID NO: 1
W3110-crp-Preg3: deletion of nt 565-nt 624 from SEQ ID NO: 1

W3110 strains having a shortened crp promoter were produced by using homologous recombination to replace the kan-sacB cassette of the strain W3110-crp::kan-sacB with a DNA fragment containing the modified promoter sequence. DNA fragments having the modified promoter sequences were produced in a known manner by fusion PCR (so-called OE-PCR, short for “Overlap Extension PCR”) from two PCR products defining the modified promoter. To produce the PCR products described in what follows, use was made in each case of the “Phusion™ High-Fidelity” DNA polymerase (Thermo Scientific™) in accordance with the manufacturer's instructions.

The following primers were used:
crp-13f (SEQ ID NO: 7): corresponds to nt 160-180 in SEQ ID NO: 1.
crp-14r (SEQ ID NO: 8): corresponds to nt 545-564 in SEQ ID NO: 1; in reverse-complementary form.
crp-17f (SEQ ID NO: 9): corresponds to nt 537-565 (nt 1 to 29 in crp-17f) and nt 866-885 (nt 30 to 49 in crp-17f) in SEQ ID NO: 1.
crp-12r (SEQ ID NO: 10): corresponds to nt 1478-1498 in SEQ ID NO: 1; in reverse-complementary form.
crp-18f (SEQ ID NO: 11): corresponds to nt 537-564 (nt 1 to 28 in crp-18f) and nt 714-734 (nt 29 to 49 in crp-18f) in SEQ ID NO: 1.
crp-19f (SEQ ID NO: 12): corresponds to nt 537-564 (nt 1 to 28 in crp-19f) and nt 676-700 (nt 29 to 53 in crp-19f) in SEQ ID NO: 1.
crp-20f (SEQ ID NO: 13): corresponds to nt 537-564 (nt 1 to 28 in crp-20f) and nt 625-644 (nt 29 to 48 in crp-20f) in SEQ ID NO: 1.

The following PCR products were produced:
PCR 1: PCR with genomic DNA from E. coli W3110 and the primers crp-13f and crp-14r resulted in the production of a 0.4 kb PCR product.
PCR 2: PCR with genomic DNA from E. coli W3110 and the primers crp-17f and crp-12r resulted in the production of a 0.65 kb PCR product.
PCR 3: PCR with genomic DNA from E. coli W3110 and the primers crp-18f and crp-12r resulted in the production of a 0.8 kb PCR product.
PCR 4: PCR with genomic DNA from E. coli W3110 and the primers crp-19f and crp-12r resulted in the production of a 0.8 kb PCR product.
PCR 5: PCR with genomic DNA from E. coli W3110 and the primers crp-20f and crp-12r resulted in the production of a 0.8 kb PCR product.
OE-PCR (Overlap Extension PCR), as described for example in Hilgarth and Lanigan, MethodsX (2020), 7:100759, resulted in the production of the following fusion PCR products:
PCR 6:1 kb fusion PCR product for production of the strain E. coli W3110-crpP-del as a result of OE-PCR of PCR 1 and PCR 2 and the primers crp-13f and crp-12r.
PCR 7:1.2 kb fusion PCR product for production of the strain E. coli W3110-crp-Preg as a result of OE-PCR of PCR 1 and PCR 3 and the primers crp-13f and crp-12r.
PCR 8:1.2 kb fusion PCR product for production of the strain E. coli W3110-crp-Preg2 as a result of OE-PCR of PCR 1 and PCR 4 and the primers crp-13f and crp-12r.
PCR 9:1.2 kb fusion PCR product for production of the strain E. coli W3110-crp-Preg3 as a result of OE-PCR of PCR 1 and PCR 5 and the primers crp-13f and crp-12r.
   Transformation of E. coli W3110-Crp::Kan-sacB x pKD46:

The fusion PCR products PCR 6, PCR 7, PCR 8 and PCR 9 were each transformed into E. coli W3110-crp::kan-sacB x pKD46 and clones were selected on LBS plates (10 g/L tryptone, 5 g/L yeast extract, 7% sucrose, 1.5% agar) without kanamycin. Only clones which no longer contained an active sacB gene were able to grow on LBS plates. These clones were seeded onto LBkan plates in order to select clones which also no longer contained an active Kan gene and which were inhibited in the presence of kanamycin in terms of their growth. One clone in each case was selected and the temperature-sensitive plasmid pKD46 removed by incubation at 42° C. Clones without the pKD46 plasmid were ampicillin-sensitive and were no longer able to grow on LBamp plates. The respective clones were designated E. coli W3110-crpP-del, E. coli W3110-crp-Preg, E. coli W3110-crp-Preg2, and E. coli W3110-crp-Preg3.

### PCR Analysis of the Transformants:

Clones having positive growth in the presence of sucrose and negative growth in the presence of kanamycin were selected and genomic DNA was obtained using a DNA isolation kit (Qiagen) from cells from cultivation in LB medium (10 g/L tryptone, 5 g/L yeast extract, 5 g/L NaCl).

Genomic DNA was used in a PCR reaction (“Phusion™ High-Fidelity” DNA polymerase, Thermo Scientific™) with the primers crp-7f and crp-8r to check whether the Kan-sacB cassette had been correctly replaced by the respective fusion PCR product. Clones having a PCR product of the expected size were selected in each case and an analysis for correct incorporation of the modified promoter sequence and an unaltered sequence of the crp cds was carried out by DNA sequencing of the PCR products (Eurofins Genomics).

The PCR products had the following expected sizes:
Strain E. coli W3110-crpP-del: 1397 nt. Deletion of nt 565-nt 865 from SEQ ID NO: 1.
Strain E. coli W3110-crp-Preg: 1549 nt. Deletion of nt 565-nt 713 from SEQ ID NO: 1.
Strain E. coli W3110-crp-Preg2: 1587 nt. Deletion of nt 565-nt 675 from SEQ ID NO: 1.
Strain E. coli W3110-crp-Preg3: 1637 nt. Deletion of nt 565-nt 624 from SEQ ID NO: 1.

### EXAMPLE 3: PRODUCTION OF CYSTEINE PRODUCTION STRAINS

Cysteine production strains (strains having deregulated cysteine biosynthesis) were produced by using the cysteine-specific production plasmid pCys (FIG. 3).

pCys is a derivative of the plasmid pACYC184-cysEX-GAPDH-ORF306 disclosed in EP 0 885 962 B1.

In addition to the origin of replication and a tetracycline resistance gene (parent vector pACYC184), the plasmid pACYC184-cysEX-GAPDH-ORF306 contains the cysEX allele, which encodes a serine O-acetyltransferase having reduced feedback inhibition by cysteine, and the efflux gene ydeD (ORF306), the expression of which is controlled by the constitutive GAPDH promoter.

Furthermore, pCys additionally contains the serA317 gene fragment, cloned after the ydeD (QRF306) efflux gene and encoding the N-terminal 317 amino acids of the SerA protein (total length of 410 amino acids). The E. coli serA Gen is disclosed in the “GenBank” gene database with the gene ID 945258. serA317 is disclosed in Bell et al. 2002 (see above, referred to therein as “NSD: 317” and encodes a serine feedback-resistant variant of 3-phosphoglycerate dehydrogenase. The expression of serA317 is controlled by the serA promoter.

The strains E. coli W3110, E. coli W3110-crp::kan-sacB, E. coli W3110-crpP-del, E. coli W3110-crp-Preg, E. coli W3110-crp-Preg2 and E. coli W3110-crp-Preg3 were each transformed with the plasmid pCys.

Plasmid-bearing transformants were selected on LBtet-agar plates (10 g/L tryptone, 5 g/L yeast extract, 5 g/L NaCl, 1.5% agar, 15 mg/L tetracycline). One clone was selected in each case. The strains used in the examples that follow were designated as follows:
E. coli W3110 x pCys,
E. coli W3110-crp::kan-sacB x pCys
E. coli W3110-crpP-del x pCys
E. coli W3110-crp-Preg x pCys
E. coli W3110-crp-Preg2 x pCys
E. coli W3110-crp-Preg3 x pCys

### EXAMPLE 4: CULTIVATION IN A SHAKE FLASK

Preculture: As a preculture for cultivation in a shake flask, 3 ml of LBtet medium (10 g/L tryptone, 5 g/L yeast extract, 10 g/L NaCl, 15 mg/L tetracycline) were inoculated with the respective cysteine production strain from Example 3 and incubated in a shaker (Infors) at 30° C. and 135 rpm for 16 h and the OD600/ml (optical density of the cultivation per ml of culture, measured at 600 nm) was determined.

Main culture: Thereafter, 300 ml Erlenmeyer flasks (baffled) containing 30 ml of SM1 medium containing 15 g/L glucose, 5 mg/L vitamin B1, 2 g/L Na thiosulfate and 15 mg/L tetracycline were inoculated with a volume of the preculture to achieve an OD600/ml of 0.05.

Composition of the SM1 medium: 12 g/L K₂HPO₄, 3 g/L KH₂PO₄, 5 g/L (NH₄)₂SO₄, 0.3 g/L MgSO₄×7H₂O, 0.015 g/L CaCl₂)×2H₂O, 0.002 g/L FeSO₄×7H₂O, 1 g/L Na₃ citrate×2H₂O, 0.1 g/L NaCl; 1 ml/L trace element solution.

Composition of the trace element solution: 0.15 g/L Na₂MoO₄×2H₂O, 2.5 g/L H₃BO₃, 0.7 g/L CoCl₂×6H₂O, 0.25 g/L CuSO₄×5H₂O, 1.6 g/L MnCl₂×4H₂O, 0.3 g/L ZnSO₄×7H₂O.

To isolate RNA for RT-PCR experiments, 30 ml batches were incubated at 30° C. and 140 rpm up to an OD600/ml of 0.5/ml (4-5 h incubation time).

To determine cysteine production, 30 ml batches were incubated at 30° C. and 140 rpm for 24 h.

For comparative analysis of the cysteine production of a WT strain having non-deregulated cysteine biosynthesis in Example 6, the strain E. coli W3110 without plasmid pCys was cultivated in the same way, but without tetracycline in all media when culturing this strain having non-deregulated cysteine biosynthesis.

### EXAMPLE 5: ANALYSIS OF CRP EXPRESSION BY RT-PCR

1) Isolation of RNA: 0.5 ml of main culture having an OD600/ml of 0.5/ml from Example 4 was admixed with 1 ml of RNA-Protect® (“Bacteria Reagent”, Qiagen) to stabilize RNA. RNA was isolated from the samples with the aid of the RNeasy RNA isolation kit (RNeasy Mini Kit, Qiagen) in accordance with the manufacturer's instructions. Depending on the strain, 8 to 10 μg of RNA were isolated from 0.5 ml of main culture. RNA concentration was determined by means of a Qubit 3.0 fluorometer from Thermo Fisher Scientific using the “Qubit™ RNA BR Assay Kit” according to the manufacturer's instructions.

2) Production of complementary DNA (cDNA): 1.5 μg of isolated RNA were converted into cDNA by reverse transcription using the QuantiNova™ Reverse Transcription Kit (Qiagen). cDNA concentration was determined by means of a Qubit 3.0 fluorometer from Thermo Fisher Scientific using the “Qubit™ dsDNA HS Assay Kit” in accordance with the manufacturer's instructions. The cDNA yield from 1.5 μg of RNA was 350-390 ng.

3) RT-PCR: Use was made of a RotorGene Q 2plex RT-PCR instrument from Qiagen, operated by the RotorGene Q control and evaluation software from the same manufacturer. Use was additionally made of the QuantiNova™ Sybr® Green PCR Kit for Real-Time PCR (Qiagen).

The expression of the crp gene and of the cysG gene as reference gene were analyzed. The reference gene cysG served for normalization as an internal standard which changes little in expression (Zhou et al. 2011 (see above)) and against which the expression of the crp gene was compared.

The following primers were used:
crp gene: primers crp-1f (SEQ ID NO: 14) and crp-2r (SEQ ID NO: 15).
cysG gene: primers cysg-1f (SEQ ID NO: 16) and cysg-2r (SEQ ID NO: 17).

What was analyzed was cDNA from the shake flask cultivation of the six strains transformed with the plasmid pCys from Example 4, with crp expression in the strain W3110 x pCys serving as a reference point for evaluating the relative expression of the crp gene (comparative strain; crp expression by the wild-type promoter) and crp expression in the other strains being compared against said reference point. For statistical evidence, each RT-PCR reaction was carried out four times identically and simultaneously as replicates (quadruplicate determination). For the cDNA of the six strains, the RT-PCR reactions were carried out at the same time in one run for the cysG reference gene and for the crp gene to be determined, which corresponded to altogether 48 RT-PCR reactions for quadruplicate determination of the expression of two genes per strain.

RT-PCR reactions: An RT-PCR reaction (final volume of 20 μl) for analysis of the expression of the crp gene was composed of 10 μl of H₂O, containing 20 ng of cDNA and 14 pmol each of the crp-specific primers crp-lf and crp-2r, and 10 ul of QuantiNova™ Sybr® Green Mastermix for Real-Time PCR (Qiagen), containing the DNA polymerase and the fluorescent dye Sybr® Green for detection of newly formed DNA. An RT-PCR reaction (final volume of 20 μl) for analysis of the expression of the cysG gene was composed of 10 μl of H₂O, containing 20 ng of cDNA and 14 pmol each of the cysG-specific primers cysg-lf and cysg-2r, and 10 μl of QuantiNova™ Sybr® Green Mastermix for Real-Time PCR (Qiagen), containing the DNA polymerase and the fluorescent dye Sybr® Green for detection of newly formed DNA.

Following an initial incubation of 2 min at 95° C., the RT-PCR program consisted of 40 cycles of 5 sec at 95° C. and 10 sec at 60° C. each. The fluorescent signal caused by binding of Sybr® Green to newly formed double-stranded DNA was registered by a detector in the RT-PCR instrument and the plot of said fluorescent signal against time was analyzed by the evaluation software.

4) Evaluation of RT-PCR: The evaluation software determined crp expression in a strain relative to crp expression in the comparative strain W3110 x pCys. The basis of the analysis was the so-called 2^{-ΔΔCT} method, the mathematical derivation of which has been described by Livak and Schmittgen 2001 (see above). For the comparative strain, this analysis yielded a 2^{-ΔΔCT} value of 1. Values>1 thus mean increased crp expression, and values<1 accordingly mean reduced crp expression.

The plot of the fluorescent signal against time was used by the evaluation software to determine the mean of the so-called CT value (CT “cycle threshold”) for each sample determined in quadruplicate and, in a first step, this was used to form the difference ΔCT between the CT values of the crp gene and of the cysG reference gene, as ΔCT=CT_{crp}−CT_{cysG}, for each strain. In a second step, the difference ΔΔCT between the ΔCT value of a strain to be analyzed and the ΔCT value of the comparative strain W3110 x pCys was formed as ΔΔCT=ΔCT−ΔCT_{W3110 x pCys}. In a third step, the ΔΔCT value was used to form the value 2^{-ΔΔCT}, which was a measure of the relative expression of the crp gene in a strain in comparison with the expression of the crp gene in the comparative strain W3110 x pCys (Wt crp promoter). Table 1 summarizes the relative crp expression in the strains studied in comparison with the expression in the comparative strain W3110 x pCys (expression normalized to a value of 1).

TABLE 1
Relative expression of the crp gene in W3110 promoter
variants in relation to the comparative strain
W3110 × pCys, as determined by RT-PCR

| | CT | CT | | | |
|---|---|---|---|---|---|
| Strain | (cysG) | (crp) | ΔCT* | ΔΔCT** | 2^{-ΔΔCT} |
| W3110 × pCys | 27.33 | 24.75 | −2.58 | 0 | 1.00 |
| W3110-crp::kan-sacB × | 27.46 | 26.46 | −1.00 | 1.58 | 0.33 |
| pCys | | | | | |
| W3110-crpP-del × pCys | 26.18 | 28.84 | 2.66 | 5.24 | 0.03 |
| W3110-crp-Preg × pCys | 27.27 | 28.95 | 1.68 | 4.26 | 0.05 |
| W3110-crp-Preg2 × pCys | 26.99 | 25.42 | −1.57 | 1.01 | 0.50 |
| W3110-crp-Preg3 × pCys | 27.15 | 24.7 | −2.45 | 0.13 | 0.91 |

*ΔCT: CT_{crp} − CT_{cysG}
**ΔΔCT: ΔCT − ΔCT_{(comparative strain W3110×pCys)}

### EXAMPLE 6: DETERMINATION OF CYSTEINE PRODUCTION FROM SUPERNATANTS FROM SHAKE FLASK CULTIVATION

After 24 h of cultivation, samples were taken from the main cultures from Example 4 and the cell density OD600/ml and the total cysteine content in the culture supernatant were determined, the colorimetric assay by Gaitonde 1967 (see above) being used for quantitative determination of cysteine. 20 μl of culture supernatant were used in 2 ml of assay. It should be borne in mind that, under the highly acidic reaction conditions, this assay does not distinguish between cysteine and the condensation product of cysteine and pyruvate, 2-methylthiazolidine-2,4-dicarboxylic acid (thiazolidine), that is described in EP 0 885 962 B1. L-cystine, which is formed by oxidation of two cysteine molecules according to equation (2), is likewise detected as cysteine in the assay by reduction with dithiothreitol in dilute solution at pH 8.0.

Table 2 contains the cell density (OD600/ml) and the cysteine content (cysteine in mg/ml) of the main cultures, and additionally specific cysteine production in relation to cell density (cysteine in mg/OD) and specific cysteine production (cysteine in % of W3110 x pCys) in percent in relation to the comparative strain W3110 x pCys (=100%).

TABLE 2
Cysteine production in a shake flask by W3110
strains having a mmodified crp promoter

| | | | | Cysteine |
|---|---|---|---|---|
| | | | | content in |
| | | | | relation to |
| | | | | W3110 × |
| | | Cyste- | Cyste- | pCys |
| | | ine | ine | (0.062 |
| Strain | OD₆₀₀/ml | mg/ml | mg/OD | mg/OD) |
| W3110 × pCys | 6.0 | 0.37 | 0.062 | 100.0% |
| W3110-crp::kan-sacB × | 5.8 | 0.50 | 0.087 | 140.0% |
| pCys | | | | |
| W3110-crpP-del × pCys | 2.5 | 0.44 | 0.177 | 285.5% |
| W3110-crp-Preg × pCys | 4.9 | 1.10 | 0.223 | 359.7% |
| W3110-crp-Preg2 × pCys | 5.7 | 0.40 | 0.070 | 112.9% |
| W3110-crp-Preg3 × pCys | 5.7 | 0.47 | 0.083 | 133.9% |
| W3110 | 8.4 | 0.00 | 0.000 | 0.0% |

### EXAMPLE 7: CYSTEINE PRODUCTION IN A FERMENTER

A comparison was made between E. coli W3110 x pCys, W3110-crp::kan-sacB x pCys, W3110-crp-Preg2 x pCys and W3110-crp-Preg3 x pCys in production-scale fed-batch fermentation. The strains E. coli W3110-crpP-del x pCys and W3110-crp-Preg x pCys having the lowest crp expression (Table 1) were not studied further because of poor fermenter growth.

### Preculture 1:

20 ml of LBtet medium were inoculated with the respective strain in a 100 ml Erlenmeyer flask and incubated on a shaker (150 rpm, 30° C.) for 7 h.

### Preculture 2:

Thereafter, the respective precultures 1 were transferred completely to 100 ml of SM1 medium supplemented with 5 g/L glucose, 5 mg/L vitamin B1 and 15 mg/L tetracycline (for the composition of SM1 medium, see Example 4).

The cultures were shaken in Erlenmeyer flasks (1 L volume) at 30° C. for 17 h at 150 rpm (Infors incubator shaker). Following this incubation, the cell density OD600/ml was between 3 and 5.

Main culture:

Fermentation was carried out in a “DASGIP® Parallel Bioreactor System for Microbiology” fermenter from Eppendorf. Culture vessels with a total volume of 1.8 L were used. The fermentation medium (900 ml) contained 15 g/L glucose, 10 g/L tryptone (Difco), 5 g/L yeast extract (Difco), 5 g/L (NH₄)₂SO₄, 1.5 g/L KH₂PO₄, 0.5 g/L NaCl, 0.3 g/L MgSO₄×7H₂O, 0.015 g/L CaCl₂)×2H₂O, 0.075 g/L FeSO₄×7H₂O, 1 g/L Na₃ citrate×2H₂O and 1 ml of trace element solution (see Example 6), 0.005 g/L vitamin B1 and 15 mg/L tetracycline.

The pH in the fermenter was initially adjusted to 7.0 by pumping in a 25% NH₄OH solution. During the fermentation, the pH was maintained at a value of 7.0 by automatic correction with 25% NH₄OH.

For inoculation, 100 ml of preculture 2 were pumped into the fermenter vessel. The initial volume was therefore about 1 L. The cultures were initially stirred at 400 rpm and aerated with compressed air sterilized via a sterile filter at an aeration rate of 2 vvm (vvm: input of compressed air into the fermentation batch specified in liters of compressed air per liter of fermentation volume per minute). Under these starting conditions, the oxygen probe was calibrated to 100% saturation prior to inoculation.

The target value for the O₂ saturation during the fermentation was set to 30%. After the O₂ saturation had fallen below the target value, a regulation cascade was started in order to bring the O₂ saturation back up to the target value. This involved first increasing the gas supply continuously (to a maximum of 5 vvm) and then increasing the stirring speed continuously (to a maximum of 1500 rpm).

The fermentation was carried out at a temperature of 30° C. After a fermentation time of 2 h, a sulfur source in the form of a sterile 60% (w/v) stock solution of sodium thiosulfate x 5H₂O was fed in at a rate of 1.5 ml per hour.

Once the glucose content in the fermenter had fallen from an initial 15 g/L to approx. 2 g/L, a 56% (w/w) glucose solution was continuously metered in. The feeding rate was adjusted such that the glucose concentration in the fermenter no longer exceeded 2 g/L from then on. Glucose was determined using a glucose analyzer from YSI (Yellow Springs, Ohio, USA).

The fermentation time was 48 h. Thereafter, samples were taken from the fermentation batch and separate determination of the content of L-cysteine and the derivatives derived therefrom in the culture supernatant (primarily L-cysteine and thiazolidine) and in the precipitate (L-cystine) was carried out. For this purpose, use was made of the colorimetric assay by Gaitonde 1967 in each case (see above). The L-cystine present in the precipitate first had to be dissolved in 8% (v/v) hydrochloric acid before it could be quantified in the same way. Lastly, the total amount of cysteine was determined as the sum total of cysteine in the pellet and in the supernatant.

As summarized in Table 3, the cell density OD600/ml of the strains studied was comparable. By contrast, the amount of cysteine produced per volume (in g/L) was higher in W3110-crp::kan-sacB x pCys, W3110-crp-Preg2 x pCys and W3110-crp-Preg3 x pCys than in the control strain W3110 x pCys having an unmodified crp promoter.

TABLE 3
Cell density and total cysteine content after
a culture time of 48 h in a fermenter

| | Cell density | Cysteine |
|---|---|---|
| Strain | OD₆₀₀/ml | (g/L) |
| W3110 × pCys | 67 | 15.9 |
| W3110-crp::kan-sacB × pCys | 70.4 | 31.7 |
| W3110-crp-Preg2 × pCys | 60 | 41 |
| W3110-crp-Preg3 × pCys | 84 | 33.6 |

1-13. (canceled)

14. A microorganism strain having a deregulated cysteine biosynthesis pathway that is thereby suitable for fermentative production of at least one substance selected from L-cysteine, L-cystine and thiazolidine, characterized in that the relative expression of the crp gene is reduced in relation to the expression of the crp gene having a wild-type promoter sequence as a result of mutation of the crp promoter sequence.

15. The microorganism strain as claimed in claim 14, wherein the amino acid sequence of the Crp protein is nonmutated.

16. The microorganism strain as claimed in claim 14, wherein the mutation in the crp promoter sequence comprises at least one deletion or insertion.

17. The microorganism strain as claimed in claim 14, wherein at least nt 565-624 is deleted from the crp promoter sequence specified in SEQ ID NO: 1 nt 565-865.

18. The microorganism strain as claimed claim 14, wherein the crp promoter sequence is completely deleted.

19. The microorganism strain as claimed in claim 14, wherein the microorganism strain is a strain from the Enterobacteriaceae or Corynebacteriaceae family.

20. The microorganism strain as claimed in claim 14, wherein the microorganism strain is selected from the group consisting of Escherichia coli, Pantoea ananatis and Corynebacterium glutamicum.

21. The microorganism strain as claimed in claim 14, wherein the microorganism strain is a strain of the species Escherichia coli.

22. The microorganism strain as claimed in claim 14, characterized in that the expressed Crp protein is SEQ ID NO: 2.

23. A process for producing at least one compound selected from L-cysteine, L-cystine and thiazolidine, wherein a microorganism strain as claimed in claim 14 is used.

24. The process as claimed in claim 23, wherein the L-cysteine, L-cystine or thiazolidine formed is isolated.

25. The process as claimed in claim 24, wherein crp expression in the microorganism strain having a modified crp promoter sequence is reduced by at least 9% in comparison with a corresponding microorganism strain having a Wt crp promoter sequence and the yield of a substance selected from L-cysteine, L-cystine and thiazolidine in g/l is increased by at least 10% (w/v) when using the microorganism strain.

26. The process as claimed in claim 23, wherein the process is a fermentative process and the fermentation volume is at least 1 L.


SEQUENCE LISTING (17 sequences)

SEQ ID NO: 1 - DNA, 1696 bp, Escherichia coli
  gene (1)..(1696): crp genomic region
cacatcaatacgttagtggtaccgggcggcaaaatgggtctggcaatggaattaattatgctgccgctggtgcaacgattgatggaaggaaagaaaatcgagtaactctgctattacgccggataaaatactatccggcttcacaacgggatagttaagtcacgcggcaaccacttcatacgagtgagtaatattcaccgctttttccagcatcaacgccactgaacaatatttctcggcagagagatcaaccgcacgcgcaaccgctgcgtctttcaggtcgcgaccggtgacgataaaatgcagattaatgtgcgtaaacaggcgtggtgcctcttcgcggcgttcagaggtcaattttacttcacaatcgaccacatcctgacgccctttttgcaggatcgaaaccacatcgatggcactgcaaccacccgccgccatcaacaccatttccatcggacttggtgctttatcgcctgagttgccgtccattaaaatctgatgaccagaggcggattcgcccagaaaagttaacccttcgacccacttcactcgcgcttgcatttttgctactccactgcgtcaattttcctgacagagtacgcgtactaaccaaatcgcgcaacggaaggcgacctgggtcatgctgaagcgagacaccaggagacacaaagcgaaagctatgctaaaacagtcaggatgctacagtaatacattgatgtactgcatgtatgcaaaggacgtcacattaccgtgcagtacagttgatagccccttcccaggtagcgggaagcatatttcggcaatccagagacagcggcgttatctggctctggagaaagcttataacagaggataaccgcgcatggtgcttggcaaaccgcaaacagacccgactctcgaatggttcttgtctcattgccacattcataagtacccatccaagagcacgcttattcaccagggtgaaaaagcggaaacgctgtactacatcgttaaaggctctgtggcagtgctgatcaaagacgaagagggtaaagaaatgatcctctcctatctgaatcagggtgattttattggcgaactgggcctgtttgaagagggccaggaacgtagcgcatgggtacgtgcgaaaaccgcctgtgaagtggctgaaatttcgtacaaaaaatttcgccaattgattcaggtaaacccggacattctgatgcgtttgtctgcacagatggcgcgtcgtctgcaagtcacttcagagaaagtgggcaacctggcgttcctcgacgtgacgggccgcattgcacagactctgctgaatctggcaaaacaaccagacgctatgactcacccggacggtatgcaaatcaaaattacccgtcaggaaattggtcagattgtcggctgttctcgtgaaaccgtgggacgcattctgaagatgctggaagatcagaacctgatctccgcacacggtaaaaccatcgtcgtttacggcactcgttaatcccgtcggagtggcgcgttacctggtagcgcgccattttgtttcccccgatgtggcgcagactgatttatcaccccgatatcaactatgcacttcgacaaacgctggtgctatgtttgcccgtggccgttgggttaatgcttggcgaattacgattcggtctgctcttctccctcgttcctgcctgttgcaatattg

SEQ ID NO: 2 - protein, 210 aa, Escherichia coli
  PEPTIDE (1)..(210): Crp Protein
MVLGKPQTDPTLEWFLSHCHIHKYPSKSTLIHQGEKAETLYYIVKGSVAVLIKDEEGKEMILSYLNQGDFIGELGLFEEGQERSAWVRAKTACEVAEISYKKFRQLIQVNPDILMRLSAQMARRLQVTSEKVGNLAFLDVTGRIAQTLLNLAKQPDAMTHPDGMQIKITRQEIGQIVGCSRETVGRILKMLEDQNLISAHGKTIVVYGTR

SEQ ID NO: 3 - DNA, 70 bp, Artificial Sequence
  Primer
  primer_bind (1)..(70): Primer
cctgacagagtacgcgtactaaccaaatcgcgcaacggaaggcgacctggcctcacgctgccgcaagcac

SEQ ID NO: 4 - DNA, 72 bp, Artificial Sequence
  Primer
  primer_bind (1)..(72): Primer
ctgtactgcacggtaatgtgacgtcctttgcatacatgcagtacatcaatgagaacaactgttcaccgttag

SEQ ID NO: 5 - DNA, 22 bp, Artificial Sequence
  Primer
  primer_bind (1)..(22): Primer
cacatcaatacgttagtggtac

SEQ ID NO: 6 - DNA, 22 bp, Artificial Sequence
  Primer
  primer_bind (1)..(22): Primer
caatattgcaacaggcaggaac

SEQ ID NO: 7 - DNA, 21 bp, Artificial Sequence
  Primer
  primer_bind (1)..(21): Primer
tcacgcggcaaccacttcata

SEQ ID NO: 8 - DNA, 20 bp, Artificial Sequence
  Primer
  primer_bind (1)..(20): Primer
atgcaagcgcgagtgaagtg

SEQ ID NO: 9 - DNA, 49 bp, Artificial Sequence
  Primer
  primer_bind (1)..(49): Primer
cttcgacccacttcactcgcgcttgcattatggtgcttggcaaaccgca

SEQ ID NO: 10 - DNA, 21 bp, Artificial Sequence
  Primer
  primer_bind (1)..(21): Primer
ttaacgagtgccgtaaacgac

SEQ ID NO: 11 - DNA, 49 bp, Artificial Sequence
  Primer
  primer_bind (1)..(49): Primer
cttcgacccacttcactcgcgcttgcatcattgatgtactgcatgtatg

SEQ ID NO: 12 - DNA, 53 bp, Artificial Sequence
  Primer
  primer_bind (1)..(53): Primer
cttcgacccacttcactcgcgcttgcatgaaagctatgctaaaacagtcagga

SEQ ID NO: 13 - DNA, 48 bp, Artificial Sequence
  Primer
  primer_bind (1)..(48): Primer
cttcgacccacttcactcgcgcttgcatacggaaggcgacctgggtca

SEQ ID NO: 14 - DNA, 21 bp, Artificial Sequence
  Primer
  primer_bind (1)..(21): Primer
agagcacgcttattcaccagg

SEQ ID NO: 15 - DNA, 21 bp, Artificial Sequence
  Primer
  primer_bind (1)..(21): Primer
aggcccagttcgccaataaaa

SEQ ID NO: 16 - DNA, 20 bp, Artificial Sequence
  Primer
  primer_bind (1)..(20): Primer
ttgtcggcggtggtgatgtc

SEQ ID NO: 17 - DNA, 24 bp, Artificial Sequence
  Primer
  primer_bind (1)..(24): Primer
atgcggtgaactgtggaataaacg